(19) United States
(12) Patent Application Publication
(10) Pub. No.: US 2002/0155564 A1
(43) Pub. Date: Oct. 24, 2002

(54) Cloning of a high growth gene

(75) Inventors: Juan F. Medrano, Davis, CA (US); Eric Bradford, Davis, CA (US); Simon Horvat, Edinburgh (GB)

Correspondence Address: Tom Hunter, SKJERVEN MORRILL MacPHERSON LLP, Suite700, 25 Metro Drive, San Jose, CA, 95110-1349, US

(73) Assignee: The Regents of the University of California

(21) Appl. No.: 09/771,208

(22) Filed: Jan. 26, 2001

Related U.S. Application Data
(63) Continuation-in-part of application No. 08/999,477, filed on Dec. 29, 1997.

(51) Int. Cl. A01K 67/027; C07H 21/04; C12N 9/99; C12P 21/02; C12N 5/06

(52) U.S. Cl. 435/184; 800/14; 435/69.2; 435/320.1; 435/325; 536/23.2

(57) ABSTRACT

This invention pertains to the discovery of a gene, reduced expression of which results in a high growth (hg) phenotype. This invention pertains to knockout animals displaying such a high growth phenotype and to methods of screening for agents that modulate a high growth phenotype.

### CROSS-REFERENCE TO RELATED APPLICATIONS

This non-provisional application is filed under 37 C.F.R. § 1.53(b) as a continuation-in-part of co-pending U.S. patent application Ser. No. 08/999,477, filed Dec. 29, 1997, which is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with the Government support under Grant Nos. HD 00394 and HD 07205, awarded by the National Institutes of Health and Grant No. 92-37205-7840 awarded by the United States Department of Agriculture. The Government of the United States of America may have certain rights in this invention.

### FIELD OF THE INVENTION

This invention generally relates to modifying growth in animals (e.g. domestic animals), and more particularly to oligonucleotide probes useful to isolate genomic clones so as to improve growth performance and efficiency in domestic animals, such as by knocking out loci related to the control of growth or utilizing identified growth quantitative trait loci in marker-assisted selection programs with animals. The invention identifies genes that are in the pathways of apoptosis and signal transduction within cells, which are directly or indirectly involved in the processes of cell proliferation, cell growth and cell death and can be utilize to alter growth in mammals.

### BACKGROUND OF THE INVENTION

In animals, many different hormones (e.g. growth hormone, sex steroids) are known to have an important function in maintaining normal animal growth and to be effective growth promoters when administered exogenously (see, e.g. Kopchick (1991) Livestock Prod. Sci., 27:66-75). Some transgenic animals have been caused to express a growth hormone, and increased growth of such transgenic animals has been reported. Palmiter et al., (1982) Nature, 300:611-615, 1982) microinjected the male pronucleus of fertilized mouse eggs with a DNA fragment containing the promoter of the mouse metallothionein-I gene fused to the structural gene of rat growth hormone. Several of the transgenic mice developed from the genetically modified zygote exhibited a growth rate substantially higher than that of control mice. Palmiter et al. (1983)Science, 222:809-814, demonstrated that a similar enhancement of growth could be obtained in transgenic mice bearing an expressible human growth hormone gene. A like effect is observed when human growth hormone releasing factor is expressed in transgenic mice (Hammer et al. (1985) Nature, 315:413-416,. Bovine growth hormone has also been expressed in transgenic animals (McGrane et al. (1988) J. Biol. Chem., 15 263:11443-51; Kopchick et al. (1989) Brazil. J. Genetics, 12:37-54).

U.S. Pat. No. 5,350,836 (Kopchick et al., issued Sep. 27, 1994) entitled “Growth Hormone Antagonists,” describes administration of a protein that has growth-inhibitory activity in vertebrates and may be administered to mammals, such as bovines, when growth inhibition is desirable. Alternatively, a gene coding for the hormone is suggested for introduction into a prenatal form of a mammal to produce growth-inhibited animals.

A recent article discusses the biological function of a transforming growth factor β superfamily member and suggests it as a potentially useful target for genetic manipulation in cattle and other farm animals. This new member is called myostatin (“GDF-8”) and functions as a negative regulator of skeletal muscle growth. It was initially studied in gene knockout experiments in mice, followed by a report of the myostatin sequences of nine other vertebrate species and the identification of mutation in double-muscled cattle (McPherron et al. (1997) Nature, 387:83-90; McPherron and Lee (1997) Proc. Natl. Acad. Sci., USA, 94:12457-12461). In mice, myostatin knockouts were significantly larger than normal mice and showed a large increase in muscle mass. In Belgian Blue cattle a small deletion of eleven nucleotides and in Piedmontese cattle a single base pair mutation in the myostatin gene produced myostatin null animals having a characteristic increase in muscle mass known as “double muscling.”

A high growth, mutant mouse with unusually rapid weight gain is also known (Bradford and Famula (1984) Genet. Res., 44:293-308). The mutation was reported to be a segment of DNA located in mouse chromosome 10 that was deleted. (Medrano et al. (1991) Genet. Res., 58: 67-74, 1991; Medrano et al. (1992) The high growth gene (hg) in mice is located on chromosome 10 linked to Igfl. Advances in gene technology: Feeding the world in the 21st century, ” edited by W. J. Whelan et al., The 1992 Miami Bio/Technology Winter Symposium, 1:12; Horvat and Medrano (1995) Genetics, 139: 1737-1748; Horvat and Medrano (1996) Genomics, 36: 546-549) The region of mouse chromosome 10 where the high growth gene was localized is homologous to a region in human chromosome 12, cattle and pig chromosome 5, sheep chromosome 3 and chicken chromosome 1. The high growth mouse phenotype features of interest are: a 30-50% increase in growth of tissues and organs, but where growth does not result in obesity; an increase in the efficiency of conversion of feed to muscle mass; decreased growth hormone levels in pituitary and plasma; an elevated plasma level of insulin growth factor-1; and, an increased muscle mass due primarily to an increase in muscle fiber number (i.e., hyperplasia) and a moderate fiber hypertrophy.

Control of growth for higher organisms has a number of applications. For example, with domestic species the characterization of the gene or genes causing high growth phenotype should offer new ways to improve growth performance and efficiency. In some human growth disorders, it has been suggested that as yet unknown genetic factor(s) may be at work (Jones (1994) Growth Genetics and Hormones, 10: 6-10). A marker closely linked to a growth disorder would be useful in diagnosis and genetic counseling. In human medicine the development of a treatment to suppress or enhance growth in specific tissues and organs would be useful in certain disesease states.

### SUMMARY OF THE INVENTION

In one aspect of the present invention, an isolated nucleic acid molecule is provided that encodes a gene product which, is knocked-out in high growth mice. For example, a mouse cDNA is provided which is highly homologous to genes of various species, such as mouse, bovine, chicken and human. The mouse cDNA is shown as SEQ ID NO:1, which corresponds to the murine Raidd/Cradd gene. The present invention provides for cloning of this gene and biologically active fragments thereof, as well as preparation of oligonucleotide probes, or primers. These are useful in identifying molecules and pathways of growth regulation so as to improve animal growth and to design diagnostic and treatment strategies for growth disorders, and to develop genetic markers. In another aspect of the present invention, characterizing causal molecular defects in mouse models of overgrowth or dwarfism helps to identify the key genes and pathways that regulate the growth process. This invention reports the molecular basis for high growth (hg), a spontaneous mutation that causes a 30-50% increase in postnatal growth. This invention concludes that hg is an allele of the suppressor of cytokine signaling 2 (Socs2), a member of a family of regulators of cytokine signal transduction. This invention demonstrates mapping of Socs2 to the hg region, lack of Socs2 mRNA expression, a disruption of the Socs2 locus in high growth (hg) mice and a similarity of phenotypes of hg mice and Socs2^{-/-} mice generated by gene targeting. Characteristics of the hg phenotype indicate that Socs2 deficiency affects growth prenatally and postnatally most likely through deregulating the growth hormone (GH)/insulin-like growth factor I (IGF1). These results demonstrate a critical role for Socs2 in controlling growth.

Thus, in one embodiment, this invention provides an isolated nucleic acid (e.g. DNA, RNA, etc.) molecule encoding a gene product (e.g. mRNA, protein, etc.) that, when knocked out, results in a high growth (hg) phenotype. In a particularly preferred embodiment, the nucleic acid comprises a socs2 nucleotide sequence (e.g. the nucleotide sequence of SEQ ID NO9) or the complement thereof. In certain embodiments, the nucleic acid is labeled (e.g. with a detectable label) or unlabeled and can, optionally be present in an expression cassette and/or a vector (e.g. plasmid, BAC, P1 clone, cosmid, phagemid, etc.).

In another embodiment this invention provides a cell (e.g. a mammalian cell) transfected with a nucleic acid molecule encoding a gene product that, when knocked out, results in a high growth (hg) phenotype. The cell can be stably or transiently transfected. In particularly preferred embodiments, the cell transcribes an mRNA and/or expresses a polypeptide encoded by the transfected nucleic acid.

In still another embodiment, this invention provides methods of producing an animal characterized by a high growth (hg) phenotype. The disruption can be by a variety of methods including, but not limited to antisense molecules, knockout constructs, RNAi, catalytic DNAs, small organic molecules, and the like. The methods preferably involve inhibiting expression (e.g. transcription and/or translation, and/or activity) of a Socs2 gene or gene product. In particularly preferred embodiments, the disruption is by disrupting the by homologous recombination with a nucleic acid (“knockout construct”) that undergoes homologous recombination with a Socs2 gene and introduces a disruption in the Socs2 gene. Preferred knockout constructs encode one or more selectable markers (e.g. hyg, neo, etc.).

In certain embodiments, this invention provides knockout mammals (e.g., equine, a bovine, a rodent, a porcine, a lagomorph, a feline, a canine, a murine, a caprine, an ovine, and a non-human primate) comprising cells containing a recombinantly introduced disruption in a Socs2 gene, where the disruption results in the knockout mammal exhibiting decreased levels of SOCS2 protein as compared to a wild-type mammal (e.g. a mammal lacking the knockout). Preferred knockout mammals of this invention display a high growth (hg) phenotype. Preferred disruptions include, but are not limited to an insertion, a deletion, a frameshift mutation, a substitution, a stop codon, and the like. In particularly preferred embodiments, the disruption comprises an insertion of an expression cassette into the endogenous Socs2 gene. Particularly preferred expression cassettes comprise a selectable marker (e.g. a neo gene, a hyg gene, etc.) operably linked to at least one regulatory element (e.g a promoter). In certain knockout mammals of this invention, the disruption is in a somatic cell. In certain knockout mammals of this invention, the disruption is in a reproductive (e.g germ) cell. The mammal can be homozygous or heterozygous for the disrupted Socs2 gene. The knockout mammals of this invention can further comprise a second recombinantly disrupted gene (e.g a disruption that prevents the expression of a functional polypeptide from the disrupted second gene). The mammal can be heterozygous or homozygous for the second disrupted gene.

In still another embodiment, this invention provides methods of screening for an agent that modulates expression of a high growth (hg) phenotype. Preferred methods involve contacting a cell comprising a Socs2 gene with a test agent; and detecting a change in the expression or activity of a Socs2 gene product (e.g mRNA, polypeptide) as compared to the expression or activity of a Socs2 gene product in a cell that is contacted with the test agent at a lower concentration (e.g. 50% test agent, no test agent, etc.) , where a difference in the expression or activity of Socs2 in the contacted cell and the cell that is contacted with the lower concentration indicates that said agent modulates expression or is likely to modulate expression of a high growth (hg) phenotype. In preferred embodiments, the Socs2 gene product is detected by detecting Socs2 mRNA in said sample, e.g the mRNA to a probe that specifically hybridizes to a Socs2 nucleic acid. In preferred hybridization methods, the hybridizing is according to a method selected from the group consisting of a Northern blot, a Southern blot using DNA derived from the Lpin1 RNA, an array hybridization, an affinity chromatography, and an in situ hybridization. In certain embodiments, the probe is a member of a plurality of probes that forms an array of probes. In certain embodiments, the Socs2 mRNA is measured using a nucleic acid amplification reaction. In another embodiment, the Socs2 gene product is detected by detecting the level of a Socs2 protein in the biological sample, e.g. via a method such as capillary electrophoresis, a Western blot, mass spectroscopy, ELISA, immunochromatography, or immunohistochemistry. In certain embodiments, the cell is a cell cultured ex vivo. In other embodiments, the cell is in vivo (e.g. the test agent is contacted to an animal comprising a cell containing the Socs2 nucleic acid or the Socs2 protein). . In preferred embodiments, the test agent is not an antibody, and/or not a protein, and/or not a nucleic acid, and/or not an antisense molecule. Particularly preferred test agents include small organic molecules.

In still another embodiment, this invention provides methods of prescreening for an agent that alters the expression of a high growth phenotype. These methods preferably involve contacting a Socs2 nucleic acid or a Socs2 protein with a test agent; and detecting specific binding of the test agent to the Socs2 protein or nucleic acid. The methods can further comprise recording (the identity) fo test agents that specifically bind to the Socs2 nucleic acid or protein in a database of candidate agents that alter hg phenotype development. In preferred embodiments, the test agent is not an antibody, and/or not a protein, and/or not a nucleic acid, and/or not an antisense molecule. Particularly preferred test agents include small organic molecules. In certain embodiments, the detecting comprises detecting specific binding of the test agent to a Socs2 nucleic acid (e.g via Northern blot, Southern blot using DNA derived from a Socs2 RNA, an array hybridization, an affinity chromatography, an in situ hybridization, etc.). In certain embodiments, the detecting comprises detecting specific binding of said test agent to a Socs2 protein or fragment thereof (e.g. via capillary electrophoresis, Western blot, mass spectroscopy, ELISA, immunochromatography, immunohistochemistry, etc.). The test agent is contacted directly to the Socs2 nucleic acid or to the Socs2 protein or the test agent is contacted to a cell containing the Socs2 protein. The cell can be ex vivo (e.g in culture) or in vivo (e.g, the test agent is contacted to an animal comprising a cell containing the Socs2 nucleic acid or the Socs2 protein).

In still another embodiment, this invention provides an isolated nucleic acid comprising a nucleic acid selected from the group consisting of: a nucleic acid that specifically hybridizes to a nucleic acid selected from the group consisting of SEQ ID NO:2, and SEQ ID NO: 9 under stringent conditions; nucleic acid comprising a nucleic acid sequence selected from the group consisting of SEQ ID NO:2, and SEQ ID NO: 9. In certain embodiments, the nucleic acid is at least 10, preferably at least 15, more preferably at least 20, most preferably at least 25, 50, or 100 nucleotides in length.

Also provided are polypeptides encoded by these nucleic acids of fragments (e.g. immunogenic fragments) of such polypeptides. Antibodies (e.g. polyclonal, monoclonal, single chain, etc.) are also provided that specifically bind to such polypeptides or polypeptide fragments.

In still another embodiments, this invention provides a nucleic acid (e.g., a “knockout construct”) for disrupting a SOCS2 gene (Socs2). Preferred nucleic acids comprise said nucleic acid comprising SOCS2 gene sequences that undergo homologous recombination with an endogenous Socs2 gene: and a nucleic acid sequence that, when introduced into a Socs2 gene inhibits the expression (transcription and/or translation of the Socs2 gene or activity of the Socs2 polypeptide) SOCS2 gene. Particularly preferred nucleic acids , when introduced into a Socs2 gene create a disruption such as an insertion, a deletion, a frameshift mutation, or a stop codon. The disruption can comprise the insertion of an expression cassette into the endogenous Socs2 gene. Preferred expression cassettes comprise a selectable marker (e.g. neo, hyg, etc.). The nucleic acid (knockout construct) can comprise Socs2 nucleic acid sequences flanking a nucleic acid encoding a Socs2 disruption and, optionally, is present in a vector.

In still another embodiment, this invention provides an animal cell (e.g. mammal cell) comprising a recombinantly introduced disruption (e.g as described above) in an endogenous Socs2 gene (Socs2) where the disruption results in the cell exhibiting decreased levels of Socs2 protein as compared to a wild-type (e.g. unmodified) cell. Preferred cells include, but are not limted to cells from chicken, turkey, duck, goose, equine, bovine, rodent, porcine, lagomorph, feline, canine, murine, caprine, ovine, non-human primate, and the like. Particularly preferred cells include rodent cells.

### DEFINITIONS

The terms “polypeptide”, “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers. The term also includes variants on the traditional peptide linkage joining the amino acids making up the polypeptide.

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 1419), phosphorothioate (Mag et al. (1991) Nucleic Acids Res. 19:1437; and U.S. Pat. No. 5,644,048), phosphorodithioate (Briu et al. (1989) J. Am. Chem. Soc. 111 :2321, O-methylphosphoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The term “heterologous” as it relates to nucleic acid sequences such as coding sequences and control sequences, denotes sequences that are not normally associated with a region of a recombinant construct, and/or are not normally associated with a particular cell. Thus, a “heterologous” region of a nucleic acid construct is an identifiable segment of nucleic acid within or attached to another nucleic acid molecule that is not found in association with the other molecule in nature. For example, a heterologous region of a construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a host cell transformed with a construct which is not normally present in the host cell would be considered heterologous for purposes of this invention.

A “coding sequence” or a sequence that “encodes” a particular polypeptide (e.g. SOCS2, etc.), is a nucleic acid sequence which is ultimately transcribed and/or translated into that polypeptide in vitro and/or in vivo when placed under the control of appropriate regulatory sequences. In certain embodiments, the boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences. In preferred embodiments, a transcription termination sequence will usually be located 3′ to the coding sequence.

Expression “control sequences” refers collectively to promoter sequences, ribosome binding sites, polyadenylation signals, transcription termination sequences, upstream regulatory domains, enhancers, and the like, which collectively provide for the transcription and translation of a coding sequence in a host cell. Not all of these control sequences need always be present in a recombinant vector so long as the desired gene is capable of being transcribed and translated.

The transcription unit of the vectors of the invention is defined herein as the DNA sequences encoding a gene, any expression control sequences such as a promoter or enhancer, a polyadenylation element, and any other regulatory elements that may be used to modulate or increase expression, all of which are operably linked in order to allow expression of the transgene. The use of any expression control sequences which facilitate persistent expression of the gene is within the scope of the invention. Such sequences or elements may be capable of generating tissue-specific expression or may be inducible by exogenous agents or stimuli.

The phrases “hybridizing specifically to” or “specific hybridization” or “selectively hybridize to”, refer to the binding, duplexing, or hybridizing of a nucleic acid molecule preferentially to a particular nucleotide sequence under stringent conditions when that sequence is present in a complex mixture (e.g., total cellular) DNA or RNA.

The term “stringent conditions” refers to conditions under which a probe will hybridize preferentially to its target subsequence, and to a lesser extent to, or not at all to, other sequences. “Stringent hybridization” and “stringent hybridization wash conditions” in the context of nucleic acid hybridization experiments such as Southern and northern hybridizations are sequence dependent, and are different under different environmental parameters. An extensive guide to the hybridization of nucleic acids is found in Tijssen (1993) Laboratory Techniques in Biochemistry and Molecular Biology—Hybridization with Nucleic Acid Probes part I chapter 2 Overview of principles of hybridization and the strategy of nucleic acid probe assays, Elsevier, N.Y. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe.

An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, Sambrook et al. (1989) Molecular Cloning—A Laboratory Manual (2nd ed.) Vol. 1-3, Cold Spring Harbor Laboratory, Cold Spring Harbor Press, NY, (Sambrook et al.) supra for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. In general, a signal to noise ratio of 2×(or higher) than that observed for an unrelated probe in the particular hybridization assay indicates detection of a specific hybridization. Nucleic acids that do not hybridize to each other under stringent conditions are still substantially identical, if the polypeptides which they encode are substantially identical. This occurs, e.g., when a copy of a nucleic acid is created using the maximum codon degeneracy permitted by the genetic code.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

The phrase “substantially identical,” in the context of two nucleic acids or polypeptides, refers to two or more sequences or subsequences that have at least 60%, preferably 80%, most preferably 90-95% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of an algorithm that is suitable for determining percent sequence identity and sequence similarity is the BLAST algorithm, which is described in Altschul et al. (1990) J. Mol. Biol. 215: 403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Natl. Acad. Sci. USA ,90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Similarity at the protein level can also refer to the ability of a subject protein to compete with hg for binding to receptors or other interacting proteins and some (but not all) monoclonal antibodies raised against hg epitopes.

The term “biological sample” refers to sample is a sample of biological tissue, cells, or fluid that, in a healthy and/or pathological state, contains a nucleic acid or polypeptide that is to be detected according to the assays described herein. Such samples include, but are not limited to, cultured cells, primary cell preparations, sputum, amniotic fluid, blood, tissue or fine needle biopsy samples, urine, peritoneal fluid, and pleural fluid, or cells therefrom. Biological samples may also include sections of tissues (e.g., frozen sections taken for histological purposes). Although the sample is typically taken from a human patient, the assays can be used to detect gugu nucleic acids or proteins in samples from any mammal, such as dogs, cats, sheep, cattle, and pigs, etc. The sample may be pretreated as necessary by dilution in an appropriate buffer solution or concentrated, if desired. Any of a number of standard aqueous buffer solutions, employing one of a variety of buffers, such as phosphate, Tris, or the like, at physiological pH can be used.

The term “test agent” refers to an agent that is to be screened in one or more of the assays described herein. The agent can be virtually any chemical compound. It can exist as a single isolated compound or can be a member of a chemical (e.g. combinatorial) library. In a particularly preferred embodiment, the test agent will be a small organic molecule.

The term “small organic molecules” refers to molecules of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The term “conservative substitution” is used in reference to proteins or peptides to reflect amino acid substitutions that do not substantially alter the activity (specificity or binding affinity) of the molecule. Typically, conservative amino acid substitutions involve substitution one amino acid for another amino acid with similar chemical properties (e.g. charge or hydrophobicity). The following six groups each contain amino acids that are typical conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

As used herein, an “antibody” refers to a protein or glycoprotein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well characterized fragments produced by digestion with various peptidases. Thus, for example, pepsin digests an antibody below (i.e. toward the Fc domain) the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region thereby converting the (Fab′)₂ dimer into an Fab′ monomer. The Fab′ monomer is essentially a Fab with part of the hinge region (see, Paul (1993) Fundamental Immunology, Raven Press, N.Y. for a more detailed description of other antibody fragments). While various antibody fragments are defined in terms of the digestion of an intact antibody, one of skill will appreciate that such fragments may be synthesized de novo either chemically, by utilizing recombinant DNA methodology, or by “phage display” methods (see, e.g., Vaughan et al. (1996) Nature Biotechnology, 14(3): 309-314, and PCT/US96/10287). Preferred antibodies include single chain antibodies, e.g, single chain Fv (scFv) antibodies in which a variable heavy and a variable light chain are joined together (directly or through a peptide linker) to form a continuous polypeptide (see, e.g. Bird et al. (1988) Science 242: 424-426; Huston et al. (1988) Proc. Nat. Acad. Sci. USA 85: 5879-5883)

### BRIEF DESCRIPTION OF THE DRAWINGS

FIG. 1 illustrates a physical map of the high growth (“hg”) region where: (A) are polymerase chain reaction (PCR)-based markers (sequence tagged sites, STSs) from ends of clones shown non-italicized, the genetic (microsatellite) marker “D10Mit69” is italicized, and a PCR-based marker derived from an exon trapping product we hereinafter call “B308A” is typed in bold. B308A corresponds to the murine Raidd/Cradd gene; (B) are genomic DNA of control mice tested as progenitors of high growth mice, AKR/J, C3H/HeJ, C57BL/6J, and DBA/2J, with the high growth mouse line being tested being C57BL/6J-hghg; (C) are Yeast Artificial Chromosome (YAC) clones, and (D) are Bacterial Artificial Chromosome (BAC) clones.

FIG. 2 illustrates Northern blots with hybridization to mouse embryonic stages and adult mouse tissues using the candidate exon “B308A” as a probe (two upper panels), and as a control the bottom two panels are blots stripped off the “B308A” probe and reprobed with cDNA for human B-actin gene.

FIG. 3A shows the nucleotide sequence of the cDNA in the mouse clone called “B308A-6-1” (SEQ ID NO:1), which corresponds to the murine Raidd/Cradd gene.

FIG. 3B shows the protein translation of the B308A-6-1 coding sequence (SEQ ID NO:4), which corresponds to the murine Raidd/Cradd gene.

FIG. 4 shows the nucleotide sequence of the original consensus B308 exon that was isolated, where polymorphism (A or T) found in one clone is indicated by a bold underlined T in position 286, and where primers used with this sequence are indicated with arrow lines (SEQ ID NO:2).

FIG. 5 illustrates a bovine fragment of the hg gene (SEQ ID NO:3) obtained with PCR primers of the cDNA mouse clone corresponding to the Raidd/Cradd gene.

FIG. 6 shows a diagram of a gene knock-out experiment for identification of the high growth gene, or locus.

FIG. 7 illustrates the identification of the high growth by gene addition.

FIG. 8 shows a Northern-blot analysis (Sambrook et al. (1989) Molecular Cloning—A Laboratory Manual (2nd ed.) Vol. 1-3, Cold Spring Harbor Laboratory, Cold Spring Harbor Press, NY; and Current Protocols in Molecular Biology) of Socs2 in the control (+, C57BLJ6J) and high growth (HG, C57BL/6J-hghg) mice demonstrating lack of Socs2 mRNA in the HG mice. The blot of total RNA (10 μg) was first probed with the Socs2 cDNA (EST clone, IMAGE ID 408909) and then reprobed with the human β-actin cDNA;

FIG. 9A illustrates the Genomic structure of the wild type Socs2 locus (+) and its disruption in the high growth (HG) mice. The murine Socs2 locus is shown with three exons and the coding region (black boxes). A 13907-bp sequence (SEQ ID NO: 9) encoding the Socs2 locus has been submitted to GenBank (accession No. AF292933); the number above each restriction enzyme site (H, HindIII; P, PvuII) relates to a position in this sequence. The deletion breakpoint in HG mice is marked with *. The probes for Southern analysis (FIG. 9B) encompassed the following nucleotides (nt) in the sequence (Genbank accession No. AF292933): Exon 2 probe (nt 4501-4639), Exon 3 probe (nt 6929-7101), and the 3′-Socs2 probe (nt 11185-11813). PCR assays I-VI (FIG. 9A) used for fine mapping of a deletion breakpoint in the HG mice utilized the same 5′ primer (nt 5089-5108) and different 3′ primers: nt 5421-5440 (I), nt 5465-5484 (II), nt 5511-5530 (III), nt 5558-5578 (IV), nt 5626-5645 (V), and nt 5671-5690 (VI). PCR assays I and II amplify in +, HG and BAC clone 520L19 DNAs (Horvat and Medrano (1998) Genomics 54: 159-164) whereas III-VI amplify in + and BAC clone 520L19 but not in HG, which maps a deletion breakpoint to intron 2 between nt 5485 and 5510. FIG. 9B shows a Southern analysis (Sambrook, supra) of the Socs2 region using 5 μg of HindIII or PvulI-digested genomic DNA of control (+, C57BL/6J) and high growth (HG, C57BL/6J-hghg) mice. On the left of each blot are the sizes of fragments (kb) that are of expected size based on the genomic sequence (FIG. 9A). Additional bands (*) hybridizing to Exon 2 and Exon 3 probes that appear in both control and HG DNA are most likely derived from the Socs2 pseudogene(s) (Metcalf, et al. (2000) Nature, 405: 1069-1073). The Exon 3 probe and the 3′-Socs2 probe hybridized to expected-size bands in control but not in HG mice demonstrating that these regions are deleted in HG. In contrast, Exon 2 probe hybridizes to a PvuII fragment (nt 2513-4678) of predicted size (2.165 kb) in both control and HG DNA showing that this fragment is retained in the HG genome. However, the exon 2 probe creates a HindIII fragment (marked with an arrowhead) of higher molecular weight (˜7 kb) in HG than in control mice (nt 3046-5976, 2.93 kb). This indicates that the HindII1 site (nt 3046) is retained in HG mice but the downstream HindIII site in intron 2 (nt 5976) is deleted, demonstrating that the deletion breakpoint is located in intron 2.

FIG. 10 shows a map of markers typed in the C57-hg/hg×CAST F₂ cross. Underlined markers were added in significant chromosomal regions identified through ANOVA. Markers in bold italics were also typed in the +/+ mice of the F₂ cross.

FIG. 11 shows sex-adjusted means and standard errors for selected traits and markers that showed significant two-way interactions with hg. Mice were classified based on their genotype at the markers closest to a detected QTL. Symbols above the C57/CAST and CAST/CAST bars indicate the significance of the contrasts: [C57/CAST−½×(C57/C57+CAST/CAST)] and [C57/C57−CAST/CAST], respectively (NS: non significant, *:p<0.05, **:p<0.01, ***:P<0.001).

FIGS. 12A and 12B show LOD score plots for G29, carcass protein, carcass ash, and femur length adjusted for the effects of sex and age, on chromosome 2 of hg/hg (FIG. 12A) and +/+ (FIG. 12B) F₂ mice. The genome-wide and chromosome significance thresholds are shown as dotted lines for hg/hg and +/+ mice respectively. Markers used in the analysis are shown below the horizontal axis. Peak LOD scores for QTL are identified with a horizontal line.

FIG. 13 is a diagram showing the breakpoints of the hg deletion and the genes (Socs2/Cish2, Raidd/Cradd and Vespr) that are included in the ˜650 kb high growth genomic region in mouse chromosome 10.

FIG. 14 shows an mRNA Northern blot showing the lack of expression of Socs2/Cish2 and Raidd/Cradd in various tissues (L, liver; B, brain, K, kidney; H, heart; Lu, lung; M, muscle; T, testis; E, 13d. embryo) in high growth (hg/hg) mice and the positive expression of Vespr in comparison to control mice (+/+).

### DETAILED DESCRIPTION

This invention pertains to the identification and isolation of a gene that regulates body size in mammals. More particularly this invention relates to the discovery of a gene, designated herein as hg or Socs2, when downregulated or knocked-out, results in a high growth (hg) phenotype. Specifically it is demonstrated herein that the hg phenotype is characterized by a particular Socs2 allele, designated herein as Socs2^{hg}.

Since an hg clone B308A-6-1 corresponding to the murine Raidd/Cradd gene identified herein, is highly conserved across species, it is possible to use conserved regions of B308A-6-1 to create probes for mapping the homologous region of hg in other species and to identify chromosome rearrangements that include the Socs2 gene in other species. For example, it is possible to use these regions of high homology to isolate the other species' hg genes by high or medium stringency hybridization, or by the polymerase chain reaction. One is able to isolate, by polymerase chain reaction, a fragment of DNA coding for hg or hg family members when using primers of degenerate sequence. Thus, using the mouse Socs2 gene identified herein, other mammalian Socs2 genes are readily identifiable. Indeed, GenBank Accession Number AF132441 provides a partial cds for the Homo sapiens suppressor of cytokine signalling-2 SOCS-2 gene and using these sequences full-length genes are readily available.

It is demonstrated herein that downregulation (e.g. via a knockout) of the Socs2 gene produces the high-growth (hg) phenotype, a phenotype characterized by a typical 30-50% increase in growth of tissues and organs, where the growth does not result in obesity. The phenotype is also characterized by an increase in the efficiency of conversion of feed to muscle mass; decreased growth hormone levels in pituitary and plasma; an elevated plasma level of insulin growth factor-1, and, an increased muscle mass due, primarily, to an increase in muscle fiber number (i.e., hyperplasia) and a moderate fiber hypertrophy.

Thus, in one embodiment, this invention provides methods for creating a high-growth (hg) phenotype mammal. The methods involve inhibiting expression of an hg nucleic acid (e.g Socs2) identified herein. Also provided are the high-growth phenotype animals themselves.

Having discovered that Socs2 inhibition results in a high growth (hg) phenotype, this invention also provides methods of screenign for agents that modulate (increase or decrease) the hg phenotype. Such methods typically involve screening for agents that upregulate or downregulate Socs2 transcription or translation or Socs2 polypeptide actigvity.

In various embodiements, this invention also provides for nucleic acids whose reduced expression provides an hg phenotype, for proteins or protein fragments encoded by such nucleic acids, and for antibodies that specifically bind such proteins or protein fragments.

The hg chromosomal region identified herein show synteny of hg with the chromosomal regions of a number of other species: The Raidd/Cradd gene (located within the hg deletion) is mapped to an interval of 100 to 103 cM from the top of the genetic map of human chromosome 12, and to 12q21.33-q23.1 in the cytogenetic map (see, also Horvat and Medrano (1998) Genomics 54: 159-164). This positioning of hg identifies a known region of synteny conservation of several human and mouse chromosome 10 loci flanking the hg region (Wakefield and Graves (1996) Mamm. Genome 7: 715-716; Archibald et al. (1999) Bioinformatics for comparative genomics in farmed and domestic animals. Plant/Animal Genome Conference (PAG-VII), San Diego, Calif., TCAGDB database; Aleyasin and Barendse (1999) Amer. Genet. Assoc. 90: 537-542) that correspond to bovine and pig chromosome 5, sheep chromosome 3, and chicken chromosome 1, suggesting that a counterpart or alleles of hg may be found in these species. Also, Raidd/Cradd, has been mapped by in-situ hybridization to chicken chromosome 1 (Smith et al. (2000) Mamm Genome 11: 706-709) corresponding to the approximate location of a growth QTL in broilers (Groenen et al. (1997) Anim. Biotechnology 8: 41-46). In addition, Stone et al. (1999) J. Anim. Sci. 77: 1379-1384, reported a QTL affecting carcass traits in cattle chromosome 5, in the homologous region to hg. No candidate genes have been proposed to date for any of these phenotypes. Without being bound by a particular theory, it is believed the hg gene of this invention finds homologs in these regions in humans and livestock.

A notable characteristic of the high growth phenotype is the consistently high level of plasma IGF-1 in growing and mature mice (Medrano et al. (1991) Genetical Research 58: 67-74; Reiser et al. (1996) Amer. J. Physiol. 40: R696-R703; Corva and Medrano (2000) Physiological Genomics 3: 17-23). IGF-1 is a growth factor with multiple functions is believed to, directly or indirectly, have an effect on cell differentiation and proliferation, and play a role as an anti-apoptotic factor (Stewart and Rotwein (1996) Phys. Rev. 76: 1005-1026). We have seen alteration in myogenesis of HG mice that may also be related to IGF-1 levels. This emphasizes the importance a gene, like Socs2/Cish2 that determines the HG phenotype has in relation to meat producing animals. In addition to growth related effects, we have observed that HG mice have a finer and denser fur. There is extensive data indicating that IGF-1 has a stimulatory effect on the activity of the hair follicle. For example, overexpression of Igf-1 in the skin of transgenic mice resulted in increased vibrissa growth (Su et al. (1999) J Invest Dermatol 112: 245-248), and a similar effect was elicited for wool growth in transgenic sheep (Damak et al. (1996) Biotechnology 14: 185-188). In humans, IGF-1 has been shown to regulate the growth of hair follicles in vitro (Philpott et al. (1994) J Invest Dematol 102: 857-861). Therefore, understanding the effect of the Socs2/Cish2 gene involved in the regulation of IGF-1 in HG mice may be useful to manipulate the levels of this hormone that may affect not only growth, but fur or wool production in economically important animals.

The identification of allelic differences in the Socs2/Cish2 gene, e.g., as described herein, is useful for selecting faster-growing and more efficient animals within breeds. Paternal lines can be developed for crossbreeding schemes carrying specific natural alleles or even artificially created null variants (e.g. knock-outs) of the hg gene. The progeny of such matings can then be marketed at early ages. The increased growth rate and feed efficiency of HG would also be a very desirable trait to express in cultured fish, like sturgeon, that have a long developmental period and slow growth rate.

Using the teaching provided herein, novel strategies exist for the manipulation of growth by altering the expression of Socs2/Cish2, such as the reduction or elimination of the gene products. This technology can be used in a wide variety of contexts including, but not limited to: A) The production of germ-line knockout animals (e.g in in chickens that have a short and intense growth period), B) The regulation of gene expression at specific times in development, e.g., by engineering zinc finger proteins as designer transcription factors in transgenic animals (see, e.g., Kang and Kim (2000) J. Biol. Chem., 275: 8742-8748), C) The creation of knockouts targeted to specific tissues, e.g. using ribozyme/antisense-mediated transgenesis or by somatic cell transfer to specific organs like the mammary gland (e.g if the growth of mammary cells is stimulated at a specific time in the development of lactation, it is milk production in cows (or other mammals) can be increased or the animals can be maintained longer in a lactating stage), and D) The use of pharmacological agents or antibodies against a protein coded by Socs2/Cish2 or to another protein in the metabolic pathway at specific stages of development of a growing or finishing animal can be useful to increase growth for a period of days in beef-cattle arriving to a feedlot, or to stimulate the growth of pigs, sheep, goats, chicken, or fish. These applications are intended to be illustrative and not limiting.

I. Inhibition of Socs2 to produce a high-growth phenotype

It is demonstrated herein that inhibition of Socs2 produces a high-growth (hg) phenotype mammal and this invention provides for mammals showing inhibited Socs2 expression. Such high-growth animals are useful in a number of contexts. For example, they provide useful organism for the study of pathologies associated with altered growth regulation, they also provide animals with a faster growth rate and increased feed conversion effciciency for the accreation of lean body mass (i.e., more consumable biomass at a lower cost per animal). Socs2 expression can be inhibited using a wide variety of approaches that include, but are not limited to antisense molecules, Socs2-specific ribozymes, Socs2-specific catalytic DNAs, Socs2-specific RNAi, intrabodies directed against Socs2 proteins, “gene therapy” approaches that knock out Socs2, and small organic molecules that inhibit Socs2 expression/overexpression or block a receptor that is required to induce Socs2.

A) Antisense approaches

Socs2 gene regulation can be downregulated or entirely inhibited by the use of antisense molecules. An “antisense sequence or antisense nucleic acid” is a nucleic acid that is complementary to the coding Socs2 mRNA nucleic acid sequence or a subsequence thereof. Binding of the antisense molecule to the Socs2 mRNA interferes with normal translation of the Socs2 polypeptide.

Thus, in accordance with preferred embodiments of this invention, preferred antisense molecules include oligonucleotides and oligonucleotide analogs that are hybridizable with Socs2 messenger RNA. This relationship is commonly denominated as “antisense.” The oligonucleotides and oligonucleotide analogs are able to inhibit the function of the RNA, either its translation into protein, its translocation into the cytoplasm, or any other activity necessary to its overall biological function. The failure of the messenger RNA to perform all or part of its function results in a reduction or complete inhibition of expression of Socs2 polypeptides.

In the context of this invention, the term “oligonucleotide” refers to a polynucleotide formed from naturally-occurring bases and/or cyclofuranosyl groups joined by native phosphodiester bonds. This term effectively refers to naturally-occurring species or synthetic species formed from naturally-occurring subunits or their close homologs. The term “oligonucleotide” may also refer to moieties which function similarly to oligonucleotides, but which have non naturally-occuring portions. Thus, oligonucleotides may have altered sugar moieties or inter-sugar linkages. Exemplary among these are the phosphorothioate and other sulfur containing species that are known for use in the art. In accordance with some preferred embodiments, at least one of the phosphodiester bonds of the oligonucleotide has been substituted with a structure which functions to enhance the ability of the compositions to penetrate into the region of cells where the RNA whose activity is to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with structures which are, at once, substantially non-ionic and non-chiral, or with structures which are chiral and enantiomerically specific. Persons of ordinary skill in the art will be able to select other linkages for use in the practice of the invention.

In one particularly preferred embodiment, the internucleotide phosphodiester linkage is replaced with a peptide linkage. Such peptide nucleic acids tend to show improved stability, penetrate the cell more easily, and show enhances affinity for their target. Methods of making peptide nucleic acids are known to those of skill in the art (see, e.g., U.S. Pat. Nos.: 6,015,887, 6,015,710, 5,986,053, 5,977,296, 5,902,786, 5,864,010, 5,786,461, 5,773,571, 5,766,855, 5,736,336, 5,719,262, and 5,714,331).

Oligonucleotides may also include species which include at least some modified base forms. Thus, purines and pyrimidines other than those normally found in nature may be so employed. Similarly, modifications on the furanosyl portions of the nucleotide subunits may also be effected, as long as the essential tenets of this invention are adhered to. Examples of such modifications are 2′-O-alkyl- and 2′-halogen-substituted nucleotides. Some specific examples of modifications at the 2′ position of sugar moieties which are useful in the present invention are OH, SH, SCH₃, F, OCH₃, OCN, O(CH₂)[n]NH₂ or O(CH₂)[n]CH₃, where n is from 1 to about 10, and other substituents having similar properties.

Such oligonucleotides are best described as being functionally interchangeable with natural oligonucleotides or synthesized oligonucleotides along natural lines, but which have one or more differences from natural structure. All such analogs are comprehended by this invention so long as they function effectively to hybridize with messenger RNA of Socs2 to inhibit the function of that RNA.

The oligonucleotides in accordance with this invention preferably comprise from about 3 to about 50 subunits. It is more preferred that such oligonucleotides and analogs comprise from about 8 to about 25 subunits and still more preferred to have from about 12 to about 20 subunits. As will be appreciated, a subunit is a base and sugar combination suitably bound to adjacent subunits through phosphodiester or other bonds. The oligonucleotides used in accordance with this invention can be conveniently and routinely made through the well-known technique of solid phase synthesis. Equipment for such synthesis is sold by several vendors (e.g. Applied Biosystems). Any other means for such synthesis may also be employed, however, the actual synthesis of the oligonucleotides is well within the talents of the routineer. It is also will known to prepare other oligonucleotide such as phosphorothioates and alkylated derivatives.

B) Catalytic RNAs and DNAs

1) Ribozymes

In another approach, Socs2 expression can be inhibited by the use of ribozymes. As used herein, “ribozymes” include RNA molecules that contain antisense sequences for specific recognition, and an RNA-cleaving enzymatic activity. The catalytic strand cleaves a specific site in a target (Socs2) RNA, preferably at greater than stoichiometric concentration. Two “types” of ribozymes are particularly useful in this invention, the hammerhead ribozyme (Rossi et al. (1991) Pharmac. Ther. 50: 245-254) and the hairpin ribozyme (Hampel et al. (1990) Nucl. Acids Res. 18: 299-304, and U.S. Pat. No. 5,254,678).

Because both hammerhead and hairpin ribozymes are catalytic molecules having antisense and endoribonucleotidase activity, ribozyme technology has emerged as a potentially powerful extension of the antisense approach to gene inactivation. The ribozymes of the invention typically consist of RNA, but such ribozymes may also be composed of nucleic acid molecules comprising chimeric nucleic acid sequences (such as DNA/RNA sequences) and/or nucleic acid analogs (e.g., phosphorothioates).

Accordingly, within one aspect of the present invention ribozymes are described that have the ability to inhibit Socs2 expression. Such ribozymes may be in the form of a “hammerhead” (for example, as described by Forster and Symons (1987) Cell 48: 211-220,; Haseloff and Gerlach (1988) Nature 328: 596-600; Walbot and Bruening (1988) Nature 334: 196; Haseloff and Gerlach (1988) Nature 334: 585) or a “hairpin” (see, e.g. U.S. Pat. No. 5,254,678 and Hampel et al., European Patent Publication No. 0 360 257, published Mar. 26, 1990), and have the ability to specifically target, cleave and Socs2 nucleic acids.

The sequence requirement for the hairpin ribozyme is any RNA sequence consisting of NNNBN*GUCNNNNNN (where N*G is the cleavage site, where B is any of G, C, or U, and where N is any of G, U, C, or A) (SEQ ID NO:______). Suitable Socs2 recognition or target sequences for hairpin ribozymes can be readily determined from the Socs2 sequence. Certain appropriate sequences include, but are not limited to sequences used as targets for antisense molecules.

The sequence requirement at the cleavage site for the hammerhead ribozyme is any RNA sequence consisting of NUX (where N is any of G, U, C, or A and X represents C, U, or A) can be targeted. Accordingly, the same target within the hairpin leader sequence, GUC, is useful for the hammerhead ribozyme. The additional nucleotides of the hammerhead ribozyme or hairpin ribozyme are determined by the target flanking nucleotides and the hammerhead consensus sequence (see Ruffner et al. (1990) Biochemistry 29: 10695-10702).

Cech et al. (U.S. Pat. No. 4,987,071,) has disclosed the preparation and use of certain synthetic ribozymes which have endoribonuclease activity. These ribozymes are based on the properties of the Tetrahymena ribosomal RNA self-splicing reaction and require an eight base pair target site. A temperature optimum of 50° C. is reported for the endoribonuclease activity. The fragments that arise from cleavage contain 5′ phosphate and 3′ hydroxyl groups and a free guanosine nucleotide added to the 5′ end of the cleaved RNA. The preferred ribozymes of this invention hybridize efficiently to target sequences at physiological temperatures, making them particularly well suited for use in vivo

The ribozymes for this invention, as well as DNA encoding such ribozymes and other suitable nucleic acid molecules can be chemically synthesized using methods well known in the art for the synthesis of nucleic acid molecules. Alternatively, Promega, Madison, Wis., USA, provides a series of protocols suitable for the production of RNA molecules such as ribozymes. The ribozymes also can be prepared from a DNA molecule or other nucleic acid molecule (which, upon transcription, yields an RNA molecule) operably linked to an RNA polymerase promoter, e.g., the promoter for T7 RNA polymerase or SP6 RNA polymerase. Such a construct may be referred to as a vector. Accordingly, also provided by this invention are nucleic acid molecules, e.g., DNA or cDNA, coding for the ribozymes of this invention. When the vector also contains an RNA polymerase promoter operably linked to the DNA molecule, the ribozyme can be produced in vitro upon incubation with the RNA polymerase and appropriate nucleotides. In a separate embodiment, the DNA may be inserted into an expression cassette (see, e.g., Cotten and Birnstiel (1989) EMBO J 8(12):3861-3866; Hempel et al. (1989) Biochem. 28: 4929-4933, etc.).

After synthesis, the ribozyme can be modified by ligation to a DNA molecule having the ability to stabilize the ribozyme and make it resistant to RNase. Alternatively, the ribozyme can be modified to the phosphothio analog for use in liposome delivery systems. This modification also renders the ribozyme resistant to endonuclease activity.

The ribozyme molecule also can be in a host prokaryotic or eukaryotic cell in culture or in the cells of an organism/patient. Appropriate prokaryotic and eukaryotic cells can be transfected with an appropriate transfer vector containing the DNA molecule encoding a ribozyme of this invention. Alternatively, the ribozyme molecule, including nucleic acid molecules encoding the ribozyme, may be introduced into the host cell using traditional methods such as transformation using calcium phosphate precipitation (Dubensky et al. (1984) Proc. Natl. Acad. Sci., USA, 81: 7529-7533), direct microinjection of such nucleic acid molecules into intact target cells (Acsadi et al. (1991) Nature 352: 815-818), and electroporation whereby cells suspended in a conducting solution are subjected to an intense electric field in order to transiently polarize the membrane, allowing entry of the nucleic acid molecules. Other procedures include the use of nucleic acid molecules linked to an inactive adenovirus (Cotton et al. (1990) Proc. Natl. Acad. Sci., USA, 89 :6094), lipofection (Felgner et al. (1989) Proc. Natl. Acad. Sci. USA 84: 7413-7417), microprojectile bombardment (Williams et al. (1991) Proc. Natl. Acad. Sci., USA, 88: 2726-2730), polycation compounds such as polylysine, receptor specific ligands, liposomes entrapping the nucleic acid molecules, spheroplast fusion whereby E coli containing the nucleic acid molecules are stripped of their outer cell walls and fused to animal cells using polyethylene glycol, viral transduction, (Cline et al., (1985) Pharmac. Ther. 29: 69; and Friedmann et al. (1989) Science 244: 1275), and DNA ligand (Wu et al (1989) J. Biol. Chem. 264: 16985-16987), as well as psoralen inactivated viruses such as Sendai or Adenovirus. In one preferred embodiment, the ribozyme is introduced into the host cell utilizing a lipid, a liposome or a retroviral vector.

When the DNA molecule is operatively linked to a promoter for RNA transcription, the RNA can be produced in the host cell when the host cell is grown under suitable conditions favoring transcription of the DNA molecule. The vector can be, but is not limited to, a plasmid, a virus, a retrotransposon or a cosmid. Examples of such vectors are disclosed in U.S. Pat. No. 5,166,320. Other representative vectors include, but are not limited to adenoviral vectors (e.g., WO 94/26914, WO 93/9191; Kolls et al. (1994) PNAS 91(1):215-219; Kass-Eisler et al., (1993) Proc. Natl. Acad. Sci., USA, 90(24): 11498-502, Guzman et al. (1993) Circulation 88(6): 2838-48, 1993; Guzman et al. (1993) Cir. Res. 73(6):1202-1207, 1993; Zabner et al. (1993) Cell 75(2): 207-216; Li et al. (1993) Hum Gene Ther. 4(4): 403-409; Caillaud et al. (1993) Eur. J Neurosci. 5(10): 1287-1291), adeno-associated vector type 1 (“AAV-1”) or adeno-associated vector type 2 (“AAV-2”) (see WO 95/13365; Flotte et al (1993) Proc. Natl. Acad. Sci., USA, 90(22):10613-10617), retroviral vectors (e.g., EP 0 415 731; WO 90/07936; WO 91/02805; WO 94/03622; WO 93/25698; WO 93/25234; U.S. Pat. No. 5,219,740; WO 93/11230; WO 93/10218) and herpes viral vectors (e.g., U.S. Pat. No. 5,288,641). Methods of utilizing such vectors in gene therapy are well known in the art, see, for example, Larrick and Burck (1991) Gene Therapy: Application of Molecular Biology, Elsevier Science Publishing Co., Inc., New York, N.Y., and Kreigler (1990) Gene Transfer and Expression: A Laboratory Manual, W. H. Freeman and Company, New York.

To produce ribozymes in vivo utilizing vectors, the nucleotide sequences coding for ribozymes are preferably placed under the control of a strong promoter such as the lac, SV40 late, SV40 early, or lambda promoters. Ribozymes are then produced directly from the transfer vector in vivo

2) Catalytic DNA

In a manner analogous to ribozymes, DNAs are also capable of demonstrating catalytic (e.g. nuclease) activity. While no such naturally-occurring DNAs are known, highly catalytic species have been developed by directed evolution and selection. Beginning with a population of 10¹⁴ DNAs containing 50 random nucleotides, successive rounds of selective amplification, enriched for individuals that best promote the Pb²⁺-dependent cleavage of a target ribonucleoside 3′-O—P bond embedded within an otherwise all-DNA sequence. By the fifth round, the population as a whole carried out this reaction at a rate of 0.2 min⁻¹. Based on the sequence of 20 individuals isolated from this population, a simplified version of the catalytic domain that operates in an intermolecular context with a turnover rate of 1 min⁻¹ (see, e.g, Breaker and Joyce (1994) Chem Biol 4: 223-229.

In later work, using a similar strategy, a DNA enzyme was made that could cleave almost any targeted RNA substrate under simulated physiological conditions. The enzyme is comprised of a catalytic domain of 15 deoxynucleotides, flanked by two substrate-recognition domains of seven to eight deoxynucleotides each. The RNA substrate is bound through Watson-Crick base pairing and is cleaved at a particular phosphodiester located between an unpaired purine and a paired pyrimidine residue. Despite its small size, the DNA enzyme has a catalytic efficiency (kcat/Km) of approximately 10⁹ M⁻¹ min⁻¹ under multiple turnover conditions, exceeding that of any other known nucleic acid enzyme. By changing the sequence of the substrate-recognition domains, the DNA enzyme can be made to target different RNA substrates (Santoro and Joyce (1997) Proc. Natl. Acad. Sci., USA, 94(9): 4262-4266). Modifying the appropriate targeting sequences (e.g. as described by Santoro and Joyce, supra.) the DNA enzyme can easily be retargeted to Socs2 mRNA thereby acting like a ribozyme.

C) RNAi inhibition of Socs2 expression

Post-transcriptional gene silencing (PTGS) or RNA interference (RNAi) refers to a mechanism by which double-stranded (sense strand) RNA (dsRNA) specifically blocks expression of its homologous gene when injected, or otherwise introduced into cells. The discovery of this incidence came with the observation that injection of antisense or sense RNA strands into Caenorhabditis elegans cells resulted in gene-specific inactivation (Guo and Kempheus (1995) Cell 81: 611-620). While gene inactivation by the antisense strand was expected, gene silencing by the sense strand came as a surprise. Adding to the surprise was the finding that this gene-specific inactivation actually came from trace amounts of contaminating dsRNA (Fire et al. (1998) Nature 391: 806-811).

Since then, this mode of post-transcriptional gene silencing has been tied to a wide variety of organisms: plants, flies, trypanosomes, planaria, hydra, zebrafish, and mice (Zamore et al. (2000). Cell 101: 25-33; Gura (2000) Nature 404: 804-808). RNAi activity has been associated with functions as disparate as transposon-silencing, anti-viral defense mechanisms, and gene regulation (Grant (1999) Cell 96: 303-306).

By injecting dsRNA into tissues, one can inactivate specific genes not only in those tissues, but also during various stages of development. This is in contrast to tissue-specific knockouts or tissue-specific dominant-negative gene expressions, which do not allow for gene silencing during various stages of the developmental process (Gura (2000) Nature 404: 804-808). The double-stranded RNA is cut by a nuclease activity into 21-23 nucleotide fragments. These fragments, in turn, target the homologous region of their corresponding mRNA, hybridize, and result in a double-stranded substrate for a nuclease that degrades it into fragments of the same size (Hammond et al. (2000) Nature, 404: 293-298; Zamore et al. (2000). Cell 101: 25-33).

Double stranded RNA (dsRNA) can be introduced into cells by any of a wide variety of means. Such methods include, but are not limited to lipid-mediated transfection (e.g. using reagents such as lipofectamine), liposome delivery, dendrimer-mediated transfection, and gene transfer using a viral or bacterial vector. Where the vector expresses (transcribes) a single-stranded RNA, the vector can be designed to trasnscribe two complementary RNA strands that will then hybridize to form a double-stranded RNA.

D) Knocking out Socs2

In another approach, Socs2 can be inhibited/downregulated simply by “knocking out” the gene. Typically this is accomplished by disrupting the Socs2 gene, the promoter regulating the gene or sequences between the promoter and the gene. Such disruption can be specifically directed to Socs2 by homologous recombination where a “knockout construct” contains flanking sequences complementary to the domain to which the construct is targeted. Insertion of the knockout construct (e.g into the Socs2 gene) results in disruption of that gene. The phrases “disruption of the gene” and “gene disruption” refer to insertion of a nucleic acid sequence into one region of the native DNA sequence (usually one or more exons) and/or the promoter region of a gene so as to decrease or prevent expression of that gene in the cell as compared to the wild-type or naturally occurring sequence of the gene. By way of example, a nucleic acid construct can be prepared containing a DNA sequence encoding an antibiotic resistance gene which is inserted into the DNA sequence that is complementary to the DNA sequence (promoter and/or coding region) to be disrupted. When this nucleic acid construct is then transfected into a cell, the construct will integrate into the genomic DNA. Thus, the cell and its progeny will no longer express the gene or will express it at a decreased level, as the DNA is now disrupted by the antibiotic resistance gene.

Knockout constructs can be produced by standard methods known to those of skill in the art. The knockout construct can be chemically synthesized or assembled, e.g., using recombinant DNA methods. The DNA sequence to be used in producing the knockout construct is digested with a particular restriction enzyme selected to cut at a location(s) such that a new DNA sequence encoding a marker gene can be inserted in the proper position within this DNA sequence. The proper position for marker gene insertion is that which will serve to prevent expression of the native gene; this position will depend on various factors such as the restriction sites in the sequence to be cut, and whether an exon sequence or a promoter sequence, or both is (are) to be interrupted (i.e., the precise location of insertion necessary to inhibit promoter function or to inhibit synthesis of the native exon). Preferably, the enzyme selected for cutting the DNA will generate a longer arm and a shorter arm, where the shorter arm is at least about 300 base pairs (bp). In some cases, it will be desirable to actually remove a portion or even all of one or more exons of the gene to be suppressed so as to keep the length of the knockout construct comparable to the original genomic sequence when the marker gene is inserted in the knockout construct. In these cases, the genomic DNA is cut with appropriate restriction endonucleases such that a fragment of the proper size can be removed.

The marker gene can be any nucleic acid sequence that is detectable and/or assayable, however typically it is an antibiotic resistance gene or other gene whose expression or presence in the genome can easily be detected. The marker gene is usually operably linked to its own promoter or to another strong promoter from any source that will be active or can easily be activated in the cell into which it is inserted; however, the marker gene need not have its own promoter attached as it may be transcribed using the promoter of the gene to be suppressed. In addition, the marker gene will normally have a polyA sequence attached to the 3′ end of the gene; this sequence serves to terminate transcription of the gene. Preferred marker genes are any antibiotic resistance gene including, but not limited to neo (the neomycin resistance gene) and beta-gal (beta-galactosidase).

After the genomic DNA sequence has been digested with the appropriate restriction enzymes, the marker gene sequence is ligated into the genomic DNA sequence using methods well known to the skilled artisan (see, e.g., Berger and Kimmel, Guide to Molecular Cloning Techniques, Methods in Enzymology volume 152 Academic Press, Inc., San Diego, Calif.; Sambrook et al. (1989) Molecular Cloning—A Laboratory Manual (2nd ed.) Vol. 1-3, Cold Spring Harbor Laboratory, Cold Spring Harbor Press, NY; and Current Protocols in Molecular Biology, F. M. Ausubel et al., eds., Current Protocols, a joint venture between Greene Publishing Associates, Inc. and John Wiley & Sons, Inc., (1994) Supplement). The ends of the DNA fragments to be ligated must be compatible; this is achieved by either cutting all fragments with enzymes that generate compatible ends, or by blunting the ends prior to ligation. Blunting is done using methods well known in the art, such as for example by the use of Klenow fragment (DNA polymerase I) to fill in sticky ends.

Suitable knockout constructs have been made and used to produce Socs2 knockout mice (see, Examples herein). The knockout constructs can be delivered to cells in vivo using gene therapy delivery vehicles (e.g. retroviruses, liposomes, lipids, dendrimers, etc.) as described below. Methods of knocking out genes are well described in the literature and essentially routine to those of skill in the art (see, e.g., Thomas et al. (1986) Cell 44(3): 419-428; Thomas, et al. (1987) Cell 51(3): 503-512)1; Jasin and Berg (1988) Genes & Development 2: 1353-1363; Mansour, et al. (1988) Nature 336: 348-352; Brinster, et al. (1989) Proc Natl Acad Sci 86: 7087-7091; Capecchi (1989) Trends in Genetics 5(3): 70-76; Frohman and Martin (1989) Cell 56: 145-147; Hasty, et al. (1991) Mol Cell Bio 11(11): 5586-5591; Jeannotte, et al. (1991) Mol Cell Biol. 11(11): 557814 5585; and Mortensen, et al. (1992) Mol Cell Biol. 12(5): 2391-2395

The use of homologous recombination to alter expression of endogenous genes is also described in detail in U.S. Pat. No. 5,272,071, WO 91/09955, WO 93/09222, WO 96/29411, WO 95/31560, and WO 91/12650.

@@Production of the knockout animals of this invention is not dependent on the availability of ES cells. In various embodiments, knockout animals of this invention can be produced using methods of somatic cell nuclear transfer. In preferred embodiments using such an approach, a somatic cell is obtained from the species in which the Socs2 gene is to be knocked out. The cell is transfected with a construct that introduces a disruption in the Socs2 gene (e.g. via heterologous recombination) as described herein. Cells harboring a knocked out Socs2 gene are selected as described herein. The nucleus of such cells harboring the knockout is then placed in an unfertilized enucleated egg (e.g., eggs from which the natural nuclei have been removed by microsurgery). Once the transfer is complete, the recipient eggs contained a complete set of genes, just as they would if they had been fertilized by sperm. The eggs are then cultured for a period before being implanted into a host mammal (of the same species that provided the egg) where they are carried to term, culminating in the berth of a transgenic animal comprising a nucleic acid construct containing one or more disrupted Ttpa genes (e.g the disrupted Ttpa gene).

The production of viable cloned mammals following nuclear transfer of cultured somatic cells has been reported for a wide variety of species including, but not limited to frogs (McKinnell (1962) J. Hered. 53, 199-207), calves (Kato et al. (1998) Science 262: 2095-2098), sheep (Campbell et al. (1996) Nature 380: 64-66), mice (Wakayamaand Yanagimachi (1999) Nat. Genet. 22: 127-128), goats (Baguisi et al. (1999) Nat. Biotechnol. 17: 456-461), monkeys (Meng et al. (1997) Biol. Reprod. 57: 454-459), and pigs (Bishop et al. (2000) Nature Biotechnology 18: 1055-1059). Nuclear transfer methods have also been used to produce clones of transgenic animals. Thus, for example, the production of transgenic goats carrying the human antithrobin III gene by somatic cell nuclear transfer has been reported (Baguisi et al. (1999) Nature Biotechnology 17: 456-461).

Using methods of nuclear transfer as describe in these and other references, cell nuclei derived from differentiated fetal or adult, mammalian cells are transplanted into enucleated mammalian oocytes of the same species as the donor nuclei. The nuclei are reprogrammed to direct the development of cloned embryos, which can then be transferred into recipient females to produce fetuses and offspring, or used to produce cultured inner cell mass (CICM) cells. The cloned embryos can also be combined with fertilized embryos to produce chimeric embryos, fetuses and/or offspring.

Somatic cell nuclear transfer also allows simplification of transgenic procedures by working with a differentiated cell source that can be clonally propagated. This eliminates the need to maintain the cells in an undifferentiated state, thus, genetic modifications, both random integration and gene targeting, are more easily accomplished. Also by combining nuclear transfer with the ability to modify and select for these cells in vitro, this procedure is more efficient than previous transgenic embryo techniques.

Nuclear transfer techniques or nuclear transplantation techniques are known in the literature. See, in particular, Campbell et al. (1995) Theriogenology, 43:181; Collas et al. (1994) Mol. Report Dev., 38:264-267; Keefer et al. (1994) Biol. Reprod., 50:935-939; Sims et al. (1993) Proc. Natl. Acad. Sci., USA, 90:6143-6147; WO 94/26884; WO 94/24274, WO 90/03432, U.S. Pat. Nos. 5,945,577, 4,944,384, 5,057,420 and the like.

Having shown that disruption of the Socs2 gene produces a high-growth (hg) phenotype, and that hg animals are viable, one of skill will recognize that there are a wide number of animals including natural and transgenic animals that have other desirable phenotypes and that can be used to practice the invention by use of ES cells and/or somatic nuclear transfer. Preferred animals are mammals including, but not limited to porcine, cows, cattle, goats, sheep, canines, felines, largomorphs, rodents, murines, primates (especially non-human primates), and the like.

E) Intrabodies

In still another embodiment, Socs2 expression/activity can be inhibited by transfecting the subject cell(s) (e.g., cells of the vascular endothelium) with a nucleic acid construct that expresses an intrabody. An intrabody is an intracellular antibody, in this case, capable of recognizing and binding to a Socs2 polypeptide. The intrabody is expressed by an “antibody cassette”, containing a sufficient number of nucleotides coding for the portion of an antibody capable of binding to the target (Socs2 polypeptide) operably linked to a promoter that will permit expression of the antibody in the cell(s) of interest. The construct encoding the intrabody is delivered to the cell where the antibody is expressed intracellularly and binds to the target Socs2, thereby disrupting the target from its normal action. This antibody is sometimes referred to as an “intrabody”.

In one preferred embodiment, the “intrabody gene” (antibody) of the antibody cassette would utilize a cDNA, encoding heavy chain variable (V_{H}) and light chain variable (V_{L}) domains of an antibody which can be connected at the DNA level by an appropriate oligonucleotide as a bridge of the two variable domains, which on translation, form a single peptide (referred to as a single chain variable fragment, “sFv”) capable of binding to a target such as an Socs2 protein. The intrabody gene preferably does not encode an operable secretory sequence and thus the expressed antibody remains within the cell.

Anti-Socs2 antibodies suitable for use/expression as intrabodies in the methods of this invention can be readily produced by a variety of methods. Such methods include, but are not limited to, traditional methods of raising “whole” polyclonal antibodies, which can be modified to form single chain antibodies, or screening of, e.g. phage display libraries to select for antibodies showing high specificity and/or avidity for Socs2. Such screening methods are described above in some detail.

The antibody cassette is delivered to the cell by any of the known means. One preferred delivery system is described in U.S. Pat. No. 6,004,940. Methods of making and using intrabodies are described in detail in U.S. Pat. Nos. 6,072,036, 6,004,940, and 5,965,371.

F) Small organic molecules

In still another embodiment, Socs2 expression and/or Socs2 protein activity can be inhibited by the use of small organic molecules. Such molecules include, but are not limited to molecules that specifically bind to the DNA comprising the Socs2 promoter and/or coding region, molecules that bind to and complex with Socs2 mRNA, molecules that inhibit the signaling pathway that results in Socs2 upregulation, and molecules that bind to and/or compete with Socs2 polypeptides. Small organic molecules effective at inhibiting Socs2 expression can be identified with routine screening using the methods described herein.

The methods of inhibiting Socs2 expression described above are meant to be illustrative and not limiting. In view of the teachings provided herein, other methods of inhibiting Socs2 will be known to those of skill in the art.

G) Zinc finger proteins as designer transcription factors to activate or repress gene expression

The ability to specifically manipulate the expression of endogenous genes have wide-ranging applications for medicine and experimental and applied biology. Nature's control mechanisms of gene activation and repression center around transcription factors that function to direct the localization of enzymes to specific DNA addresses. Exploiting this fundamental principle for imposed control of gene expression involves the utilization of sequence-specific DNA-binding domains. Of the DNA-binding motifs that have been studied, the modular zinc finger DNA-binding domains of the Cys₂-His₂ type have shown the most promise for the development of a universal system for gene regulation. Design studies and phage-based selections have shown that this motif is adaptable to the recognition of a wide variety of DNA sequences, often with exquisite specificity (Segal et al. (1999) Proc. Natl. Acad. Sci., USA, 96: 2758-2763). Recently, a family of zinc finger domains has been described that is sufficient for the construction of 17 million novel proteins that bind the 5′-(GNN)₆-3′ family of DNA sequences. These domains are functionally modular and may be recombined with one another to create polydactyl proteins capable of binding 18-bp sequences with the potential for genome-specific addressing (Beerli et al. (1998) Proc. Natl. Acad. Sci., USA, 95:14628-14633).

It has been shown that transcription factors designed to bind in the transcribed regions of either erbB-2 or erbB-3 genes, which are involved in human cancers, are capable of selectively up- or down-regulating expression of their respective target gene (Beerli et al. (2000) Proc. Natl. Acad. Sci, USA, 97: 1495-1500). A number of reports describe the details of this technology (see, e.g., Beerli et al. (2000) Proc. Natl. Acad. Sci., USA, 97: 1495-1500; Wang and Pabo (1999) Proc. Natl. Acad. Sci., USA, 96: 9568-9573; Beerli et al. (1998) Proc. Natl. Acad. Sci., USA, 95: 14628-14633; Kim et al. (1997) Proc. Natl. Acad. Sci., USA, 94: 3616-3620), which essentially consists of designing a construct expressing a zinc-finger proteins capable of recognizing and binding to regulatory sequences in the promoter of a gene that will permit activation or repression of gene expression. It has been demonstrated that constructs of this kind can regulate gene expression by stable or transient transfection in various mammalian cell lines (Kang and Kim (2000) J. Biol. Chem., 275 :8742-8748).

In the present invention the Socs2 gene can be targeted using a zinc-finger designed transcription factor protein in-vivo to repress expression of the gene, e.g, in mice. These methods can also be used to provide in-vitro cell culture system, e.g. to test the effect of repressing the expression of Socs2 various mammalian organisms, tissues, and cells. In certain embodiments, the in-vitro approach can be implemented utilizing cells from various species, such a bovine or chicken cells. The in-vitro system also provides a phenotypic assay to ascertain the modulation of GH and IGF1 in relation to the expression of Socs2.

H) Modes of administration

The mode of administration of the Socs2 blocking agent depends on the nature of the particular agent. Antisense molecules, catalytic RNAs (ribozymes), catalytic DNAs, small organic molecules, RNAi, and other molecules (e.g. lipids, antibodies, etc.) used as Socs2 inhibitors may be formulated as pharmaceuticals (e.g. with suitable excipient) and delivered using standard pharmaceutical formulation and delivery methods as described below. Antisense molecules, catalytic RNAs (ribozymes), catalytic DNAs, and additionally, knockout constructs, and constructs encoding intrabodies can be delivered and (if necessary) expressed in target cells (e.g. vascular endothelial cells) using methods of gene therapy, e.g. as described below.

1) “Pharmaceutical” formulations

In order to carry out the methods of the invention, one or more inhibitors of Socs2 expression (e.g. ribozymes, antibodies, antisense molecules, small organic molecules, etc.) are administered to a cell, tissue, or organism, to induce a high growth (hg) phenotype. Various inhibitors may be administered, if desired, in the form of salts, esters, amides, prodrugs, derivatives, and the like, provided the salt, ester, amide, prodrug or derivative is suitable pharmacologically, i.e., effective in the present method. Salts, esters, amides, prodrugs and other derivatives of the active agents may be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

The Socs2 inhibitors and various derivatives and/or formulations thereof are useful for parenteral, topical, oral, or local administration, such as by aerosol or transdermally, for prophylactic and/or therapeutic treatment of undergrowth disorders or overgrowth disorders, such as cases of uncontrolled cell proliferation which are the causal factor in tumor development. The pharmaceutical compositions can be administered in a variety of unit dosage forms depending upon the method of administration. Suitable unit dosage forms, include, but are not limited to powders, tablets, pills, capsules, lozenges, suppositories, implants etc.

The Socs2 inhibitors and various derivatives and/or formulations thereof are typically combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers.

Other physiologically acceptable compounds include wetting agents, emulsifying agents, dispersing agents or preservatives which are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s). The excipients are preferably sterile and generally free of undesirable matter. These compositions may be sterilized by conventional, well known sterilization techniques.

The concentration of active agent(s) in the formulation can vary widely, and will be selected primarily based on fluid volumes, viscosities, body weight and the like in accordance with the particular mode of administration selected and the patient's needs. Typically, the active agent(s) are administered in an amount sufficient to alter expression of Socs2, i.e., an “effective amount”. Single or multiple administrations of the compositions may be administered depending on the dosage and frequency as required and tolerated by the organism or cell or tissue system. In any event, the composition should provide a sufficient quantity of the active agents of this invention to effectively alter Socs2 expression and preferably to induce or reduce an hg phenotype.

2) “Genetic” delivery methods

As indicated above, antisense molecules, catalytic RNAs (ribozymes), catalytic DNAs, RNAi, and additionally, knockout constructs, and constructs encoding intrabodies can be delivered and transcribed and/or expressed in target cells (e.g. vascular endothelial cells) using methods of gene therapy. Thus, in certain preferred embodiments, the nucleic acids encoding knockout constructs, intrabodies, antisense molecules, catalytic RNAs or DNAs, etc. are cloned into gene therapy vectors that are competent to transfect cells (such as human or other mammalian cells) in vitro and/or in vivo.

Many approaches for introducing nucleic acids into cells in vivo, ex vivo and in vitro are known. These include lipid or liposome based gene delivery (WO 96/18372; WO 93/24640; Mannino and Gould-Fogerite (1988) BioTechniques 6(7): 682-691; Rose U.S. Pat. No. 5,279,833; WO 91/06309; and Felgner et al. (1987) Proc. Natl. Acad. Sci. USA 84: 7413-7414) and replication-defective retroviral vectors harboring a therapeutic polynucleotide sequence as part of the retroviral genome (see, e.g., Miller et al. (1990) Mol. Cell. Biol. 10:4239 (1990); Kolberg (1992) J. NIH Res. 4: 43, and Cornetta et al. (1991) Hum. Gene Ther. 2: 215).

For a review of gene therapy procedures, see, e.g., Anderson, Science (1992) 256: 808-813; Nabel and Felgner (1993) TIBTECH 11: 211-217; Mitani and Caskey (1993) TIBTECH 11: 162-166; Mulligan (1993) Science, 926-932; Dillon (1993) TIBTECH 11: 167-175; Miller (1992) Nature 357: 455-460; Van Brunt (1988) Biotechnology 6(10): 1149-1154; Vigne (1995) Restorative Neurology and Neuroscience 8: 35-36; Kremer and Perricaudet (1995) British Medical Bulletin 51(1) 31-44; Haddada et al. (1995) in Current Topics in Microbiology and Immunology, Doerfler and Böhm (eds) Springer-Verlag, Heidelberg Germany; and Yu et al., (1994) Gene Therapy, 1:13-26.

Widely used vector systems include, but are not limited to adenovirus, adeno associated virus, and various retroviral expression systems. The use of adenoviral vectors is well known to those of skill and is described in detail, e.g., in WO 96/25507. Particularly preferred adenoviral vectors are described by Wills et al. (1994) Hum. Gene Therap. 5: 1079-1088.

Adeno-associated virus (AAV)-based vectors used to transduce cells with target nucleic acids, e.g., in the in vitro production of nucleic acids and peptides, and in in vivo and ex vivo gene therapy procedures are describe, for example, by West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 for an overview of AAV vectors. Lebkowski, U.S. Pat. No. 5,173,414; Tratschin et al. (1985) Mol. Cell. Biol. 5(11):3251-3260; Tratschin, et al. (1984) Mol. Cell. Biol., 4: 2072-2081; Hermonat and Muzyczka (1984) Proc. Natl. Acad. Sci. USA, 81: 6466-6470; McLaughlin et al. (1988) and Samulski et al. (1989) J. Virol, 63:03822-3828. Cell lines that can be transformed by rAAV include those described in Lebkowski et al. (1988) Mol. Cell. Biol., 8:3988-3996.

Widely used retroviral vectors include those based upon murine leukemia virus (MuLV), gibbon ape leukemia virus (GaL V), Simian Immunodeficiency virus (SIV), human immunodeficiency virus (HIV), alphavirus, and combinations thereof (see, e.g., Buchscher et al. (1992) J. Virol. 66(5) 2731-2739; Johann et al. (1992) J. Virol. 66 (5):1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al. (1994) Gene Therapy, supra; U.S. Pat. No. 6,008,535, and the like). Other suitable viral vectors include, but are not limited to herpes virus, lentivirus, and vaccinia virus.

Alone, or in combination with viral vectors, a number of non-viral vectors are also useful for transfecting cells to express constructs that block or inhibit Socs2 expression. Suitable non-viral vectors include, but are not limited to, plasmids, cosmids, phagemids, liposomes, water-oil emulsions, polethylene imines, biolistic pellets/beads, and dendrimers.

Liposomes were first described in 1965 as a model of cellular membranes and quickly were applied to the delivery of substances to cells. Liposomes entrap DNA by one of two mechanisms which has resulted in their classification as either cationic liposomes or pH-sensitive liposomes. Cationic liposomes are positively charged liposomes which interact with the negatively charged DNA molecules to form a stable complex. Cationic liposomes typically consist of a positively charged lipid and a co-lipid. Commonly used co-lipids include dioleoyl phosphatidylethanolamine (DOPE) or dioleoyl phosphatidylcholine (DOPC). Co-lipids, also called helper lipids, are in most cases required for stabilization of liposome complex. A variety of positively charged lipid formulations are commercially available and many other are under development. Two of the most frequently cited cationic lipids are lipofectamine and lipofectin. Lipofectin is a commercially available cationic lipid first reported by Phil Felgner in 1987 to deliver genes to cells in culture. Lipofectin is a mixture of N-[1-(2, 3-dioleyloyx) propyl]-N-N-N-trimethyl ammonia chloride (DOTMA) and DOPE.

DNA and lipofectin or lipofectamine interact spontaneously to form complexes that have a 100% loading efficiency. In other words, essentially all of the DNA is complexed with the lipid, provided enough lipid is available. It is assumed that the negative charge of the DNA molecule interacts with the positively charged groups of the DOTMA. The lipid:DNA ratio and overall lipid concentrations used in forming these complexes are extremely important for efficient gene transfer and vary with application. Lipofectin has been used to deliver linear DNA, plasmid DNA, and RNA to a variety of cells in culture. Shortly after its introduction, it was shown that lipofectin could be used to deliver genes in vivo. Following intravenous administration of lipofectin-DNA complexes, both the lung and liver showed marked affinity for uptake of these complexes and transgene expression. Injection of these complexes into other tissues has had varying results and, for the most part, are much less efficient than lipofectin-mediated gene transfer into either the lung or the liver.

PH-sensitive, or negatively-charged liposomes, entrap DNA rather than complex with it. Since both the DNA and the lipid are similarly charged, repulsion rather than complex formation occurs. Yet, some DNA does manage to get entrapped within the aqueous interior of these liposomes. In some cases, these liposomes are destabilized by low pH and hence the term pH-sensitive. To date, cationic liposomes have been much more efficient at gene delivery both in vivo and in vitro than pH-sensitive liposomes. pH-sensitive liposomes have the potential to be much more efficient at in vivo DNA delivery than their cationic counterparts and should be able to do so with reduced toxicity and interference from serum protein.

In another approach dendrimers complexed to the DNA have been used to transfect cells. Such dendrimers include, but are not limited to, “starburst” dendrimers and various dendrimer polycations.

Dendrimer polycations are three dimensional, highly ordered oligomeric and/or polymeric compounds typically formed on a core molecule or designated initiator by reiterative reaction sequences adding the oligomers and/or polymers and providing an outer surface that is positively changed. These dendrimers may be prepared as disclosed in PCT/US83/02052, and U.S. Pat. Nos. 4,507,466, 4,558,120, 4,568,737, 4,587,329, 4,631,337, 4,694,064, 4,713,975, 4,737,550, 4,871,779, 4,857,599.

Typically, the dendrimer polycations comprise a core molecule upon which polymers are added. The polymers may be oligomers or polymers which comprise terminal groups capable of acquiring a positive charge. Suitable core molecules comprise at least two reactive residues which can be utilized for the binding of the core molecule to the oligomers and/or polymers. Examples of the reactive residues are hydroxyl, ester, amino, imino, imido, halide, carboxyl, carboxyhalide maleimide, dithiopyridyl, and sulfhydryl, among others. Preferred core molecules are ammonia, tris-(2-aminoethyl)amine, lysine, ornithine, pentaerythritol and ethylenediamine, among others. Combinations of these residues are also suitable as are other reactive residues.

Oligomers and polymers suitable for the preparation of the dendrimer polycations of the invention are pharmaceutically-acceptable oligomers and/or polymers that are well accepted in the body. Examples of these are polyamidoamines derived from the reaction of an alkyl ester of an α,β-ethylenically unsaturated carboxylic acid or an α,β-ethylenically unsaturated amide and an alkylene polyamine or a polyalkylene polyamine, among others. Preferred are methyl acrylate and ethylenediamine. The polymer is preferably covalently bound to the core molecule.

The terminal groups that may be attached to the oligomers and/or polymers should be capable of acquiring a positive charge. Examples of these are azoles and primary, secondary, tertiary and quaternary aliphatic and aromatic amines and azoles, which may be substituted with S or O, guanidinium, and combinations thereof. The terminal cationic groups are preferably attached in a covalent manner to the oligomers and/or polymers. Preferred terminal cationic groups are amines and guanidinium. However, others may also be utilized. The terminal cationic groups may be present in a proportion of about 10 to 100% of all terminal groups of the oligomer and/or polymer, and more preferably about 50 to 100%.

The dendrimer polycation may also comprise 0 to about 90% terminal reactive residues other than the cationic groups. Suitable terminal reactive residues other than the terminal cationic groups are hydroxyl, cyano, carboxyl, sulfhydryl, amide and thioether, among others, and combinations thereof. However others may also be utilized.

The dendrimer polycation is generally and preferably non-covalently associated with the polynucleotide. This permits an easy disassociation or disassembling of the composition once it is delivered into the cell. Typical dendrimer polycation suitable for use herein have a molecular weight ranging from about 2,000 to 1,000,000 Da, and more preferably about 5,000 to 500,000 Da. However, other molecule weights are also suitable. Preferred dendrimer polycations have a hydrodynamic radius of about 11 to 60 Å., and more preferably about 15 to 55 Å. Other sizes, however, are also suitable. Methods for the preparation and use of dendrimers in gene therapy are well known to those of skill in the art and describe in detail, for example, in U.S. Pat. No. 5,661,025

Where appropriate, two or more types of vectors can be used together. For example, a plasmid vector may be used in conjunction with liposomes. In the case of non-viral vectors, nucleic acid may be incorporated into the non-viral vectors by any suitable means known in the art. For plasmids, this typically involves ligating the construct into a suitable restriction site. For vectors such as liposomes, water-oil emulsions, polyethylene amines and dendrimers, the vector and construct may be associated by mixing under suitable conditions known in the art.

II. Assays for agents that modulate Socs2 expression

As indicated above, in one aspect, this invention pertains to the discovery that Socs2 inhibition or inactivation results in an hg (high-growth) phenotype. The Socs2 gene, or gene product(s) (e.g. mRNA, protein, etc.) provide good targets to screen for new agents that modulate Socs2 expression or activity and hence the development of an hg phenotype. Thus, in one embodiment, this invention provides methods of screening for agents that modulate Socs2 expression and/or activity. The methods preferably involve detecting a change in the expression level and/or activity level of a Socs2gene or gene product (e.g. Socs2 protein) in cell(s) contacted with the test agent in question. An elevated Socs2 expression level or activity level in the presence of the agent, e.g., as compared to a negative control where the test agent is absent or at reduced concentration indicates that the agent upregulates Socs2 activity or expression. Conversely, decreased Socs2 expression level or activity level in the presence of the agent as compared to a negative control where the test agent is absent or at reduced concentration indicates that the agent down-regulates Socs2 activity or expression

Expression levels of a gene can be altered by changes in the transcription of the gene product (i.e. transcription of mRNA), and/or by changes in translation of the gene product (i.e. translation of the protein), and/or by post-translational modification(s) (e.g. protein folding, glycosylation, etc.). Thus preferred assays of this invention include assaying for level of transcribed mRNA (or other nucleic acids derived from the Socs2 gene), level of translated protein, activity of translated protein, etc. Examples of such approaches are described below.

A) Nucleic-acid based assays

1) Target molecules

Changes in expression level can be detected by measuring changes in mRNA and/or a nucleic acid derived from the mRNA (e.g. reverse-transcribed cDNA, etc.). In order to measure the Socs2 expression level it is desirable to provide a nucleic acid sample for such analysis. In preferred embodiments, the nucleic acid is found in or derived from a biological sample. The term “biological sample”, as used herein, refers to a sample obtained from an organism or from components (e.g., cells) of an organism. The sample may be of any biological tissue or fluid. Biological samples may also include organs or sections of tissues such as frozen sections taken for histological purposes.

The nucleic acid (e.g., mRNA nucleic acid derived from mRNA) is, in certain preferred embodiments, isolated from the sample according to any of a number of methods well known to those of skill in the art. Methods of isolating mRNA are well known to those of skill in the art. For example, methods of isolation and purification of nucleic acids are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

In a preferred embodiment, the “total” nucleic acid is isolated from a given sample using, for example, an acid guanidinium-phenol-chloroform extraction method and polyA+mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Frequently, it is desirable to amplify the nucleic acid sample prior to assaying for expression level. Methods of amplifying nucleic acids are well known to those of skill in the art and include, but are not limited to polymerase chain reaction (PCR, see. e.g. Innis, et al., (1990) PCR Protocols. A guide to Methods and Application. Academic Press, Inc. San Diego,), ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc).

In a particularly preferred embodiment, where it is desired to quantify the transcription level (and thereby expression) of Socs2 in a sample, the nucleic acid sample is one in which the concentration of the Socs2 mRNA transcript(s), or the concentration of the nucleic acids derived from the Socs2 mRNA transcript(s), is proportional to the transcription level (and therefore expression level) of that gene. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes.

Where more precise quantification is required, appropriate controls can be run to correct for variations introduced in sample preparation and hybridization as described herein. In addition, serial dilutions of “standard” target nucleic acids (e.g., mRNAs) can be used to prepare calibration curves according to methods well known to those of skill in the art. Of course, where simple detection of the presence or absence of a transcript or large differences of changes in nucleic acid concentration is desired, no elaborate control or calibration is required.

In the simplest embodiment, the Socs2-containing nucleic acid sample is the total mRNA or a total cDNA isolated and/or otherwise derived from a biological sample. The nucleic acid may be isolated from the sample according to any of a number of methods well known to those of skill in the art as indicated above.

2) Hybridization-based assays

Using the Socs2 sequences provided herein, detecting and/or quantifying the Socs2 transcript(s) can be routinely accomplished using nucleic acid hybridization techniques (see, e.g., Sambrook et al. supra). For example, one method for evaluating the presence, absence, or quantity of Socs2 reverse-transcribed cDNA involves a “Southern Blot”. In a Southern Blot, the DNA (e.g., reverse-transcribed Socs2 mRNA), typically fragmented and separated on an electrophoretic gel, is hybridized to a probe specific for Socs2 (or to a mutant thereof). Comparison of the intensity of the hybridization signal from the Socs2 probe with a “control” probe (e.g. a probe for a “housekeeping gene) provides an estimate of the relative expression level of the target nucleic acid.

Alternatively, the Socs2 mRNA can be directly quantified in a Northern blot. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes are used to identify and/or quantify the target Socs2 mRNA. Appropriate controls (e.g. probes to housekeeping genes) provide a reference for evaluating relative expression level.

An alternative means for determining the Socs2 expression level is in situ hybridization. In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application.

In some applications, it is necessary to block the hybridization capacity of repetitive sequences. Thus, in some embodiments, tRNA, human genomic DNA, or Cot-1 DNA is used to block non-specific hybridization.

3) Amplification-based assays

In another embodiment, amplification-based assays can be used to measure Socs2 expression (transcription) level. In such amplification-based assays, the target nucleic acid sequences (i.e., Socs2) act as template(s) in amplification reaction(s) (e.g. Polymerase Chain Reaction (PCR) or reverse-transcription PCR (RT-PCR)). In a quantitative amplification, the amount of amplification product will be proportional to the amount of template (e.g., Socs2 mRNA) in the original sample. Comparison to appropriate (e.g. healthy tissue or cells unexposed to the test agent) controls provides a measure of the Socs2 transcript level.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). One approach, for example, involves simultaneously co-amplifying a known quantity of a control sequence using the same primers as those used to amplify the target. This provides an internal standard that may be used to calibrate the PCR reaction.

One preferred internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of labeled nucleic acid (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al. (1990) Academic Press, Inc. N.Y.. The known nucleic acid sequence(s) for Socs2 are sufficient to enable one of skill to routinely select primers to amplify any portion of the gene.

4) Hybridization formats and optimization of hybridization conditions

a) Array-based hybridization formats

In one embodiment, the methods of this invention can be utilized in array-based hybridization formats. Arrays are a multiplicity of different “probe” or “target” nucleic acids (or other compounds) attached to one or more surfaces (e.g., solid, membrane, or gel). In a preferred embodiment, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays can be produced according to a wide variety of methods well known to those of skill in the art. For example, in a simple embodiment, “low density” arrays can simply be produced by spotting (e.g by hand using a pipette) different nucleic acids at different locations on a solid support (e.g a glass surface, a membrane, etc.).

This simple spotting, approach has been automated to produce high density spotted arrays (see, e.g., U.S. Pat. No: 5,807,522). This patent describes the use of an automated system that taps a microcapillary against a surface to deposit a small volume of a biological sample. The process is repeated to generate high density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays. Synthesis of high-density arrays is also described in U.S. Pat. Nos. 5,744,305, 5,800,992 and 5,445,934.

b) Other hybridization formats

As indicated above a variety of nucleic acid hybridization formats are known to those skilled in the art. For example, common formats include sandwich assays and competition or displacement assays. Such assay formats are generally described in Hames and Higgins (1985) Nucleic Acid Hybridization, A Practical Approach, IRL Press; Gall and Pardue (1969) Proc. Natl. Acad. Sci USA 63: 378-383; and John et al. (1969) Nature 223: 582-587.

Sandwich assays are commercially useful hybridization assays for detecting or isolating nucleic acid sequences. Such assays utilize a “capture” nucleic acid covalently immobilized to a solid support and a labeled “signal” nucleic acid in solution. The sample will provide the target nucleic acid. The “capture” nucleic acid and “signal” nucleic acid probe hybridize with the target nucleic acid to form a “sandwich” hybridization complex. To be most effective, the signal nucleic acid should not hybridize with the capture nucleic acid.

Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labelled probes or the like. Other labels include ligands that bind to labeled antibodies, fluorophores, chemiluminescent agents, enzymes, and antibodies that can serve as specific binding pair members for a labeled ligand.

Detection of a hybridization complex may require the binding of a signal-generating complex to a duplex of target and probe polynucleotides or nucleic acids. Typically, such binding occurs through ligand and anti-ligand interactions as between a ligand-conjugated probe and an anti-ligand conjugated with a signal.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

c) Optimization of hybridization conditions

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency to ensure hybridization and then subsequent washes are performed at higher stringency to eliminate mismatched hybrid duplexes. Successive washes may be performed at increasingly higher stringency (e.g., down to as low as 0.25 X SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be present.

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular probes of interest.

In a preferred embodiment, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.)

Methods of optimizing hybridization conditions are well known to those of skill in the art (see, e.g., Tijssen (1993) Laboratory Techniques in Biochemistry and Molecular Biology, Vol. 24: Hybridization With Nucleic Acid Probes, Elsevier, N.Y.).

Optimal conditions are also a function of the sensitivity of label (e.g., fluorescence) detection for different combinations of substrate type, fluorochrome, excitation and emission bands, spot size and the like. Low fluorescence background surfaces can be used (see, e.g., Chu (1992) Electrophoresis 13:105-114). The sensitivity for detection of spots (“target elements”) of various diameters on the candidate surfaces can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., glass, fused silica, etc.) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

d) Labeling and detection of nucleic acids

The probes used herein for detection of Socs2 expression levels can be full length or less than the full length of the Socs2 or mutants thereof. Shorter probes are empirically tested for specificity. Preferred probes are sufficiently long so as to specifically hybridize with the Socs2 target nucleic acid(s) under stringent conditions. The preferred size range is from about 20 bases to the length of the Socs2 mRNA, more preferably from about 30 bases to the length of the Socs2 mRNA, and most preferably from about 40 bases to the length of the Socs2 mRNA.

The probes are typically labeled, with a detectable label. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

A fluorescent label is preferred because it provides a very strong signal with low background. It is also optically detectable at high resolution and sensitivity through a quick scanning procedure. The nucleic acid samples can all be labeled with a single label, e.g., a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish sites of binding of the red label from those binding the green fluorescent label. Each nucleic acid sample (target nucleic acid) can be analyzed independently from one another.

Suitable chromogens which can be employed include those molecules and compounds which absorb light in a distinctive range of wavelengths so that a color can be observed or, alternatively, which emit light when irradiated with radiation of a particular wave length or wave length range, e.g., fluorescers.

Desirably, fluorescent labels should absorb light above about 300 nm, preferably about 350 nm, and more preferably above about 400 nm, usually emitting at wavelengths greater than about 10 nm higher than the wavelength of the light absorbed. It should be noted that the absorption and emission characteristics of the bound dye can differ from the unbound dye. Therefore, when referring to the various wavelength ranges and characteristics of the dyes, it is intended to indicate the dyes as employed and not the dye which is unconjugated and characterized in an arbitrary solvent.

Fluorescers are generally preferred because by irradiating a fluorescer with light, one can obtain a plurality of emissions. Thus, a single label can provide for a plurality of measurable events.

Detectable signal can also be provided by chemiluminescent and bioluminescent sources. Chemiluminescent sources include a compound which becomes electronically excited by a chemical reaction and can then emit light which serves as the detectable signal or donates energy to a fluorescent acceptor. Alternatively, luciferins can be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

The label may be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are easily added during an in vitro transcription reaction. Thus, for example, fluorescein labeled UTP and CTP can be incorporated into the RNA produced in an in vitro transcription.

The labels can be attached directly or through a linker moiety. In general, the site of label or linker-label attachment is not limited to any specific position. For example, a label may be attached to a nucleoside, nucleotide, or analogue, thereof at any position that does not interfere with detection or hybridization as desired. For example, certain Label-ON Reagents from Clontech (Palo Alto, Calif.) provide for labeling interspersed throughout the phosphate backbone of an oligonucleotide and for terminal labeling at the 3′ and 5′ ends. As shown for example herein, labels can be attached at positions on the ribose ring or the ribose can be modified and even eliminated as desired. The base moieties of useful labeling reagents can include those that are naturally occurring or modified in a manner that does not interfere with the purpose to which they are put. Modified bases include but are not limited to 7-deaza A and G, 7-deaza-8-aza A and G, and other heterocyclic moieties.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe-CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

B) Polypeptide-based assays

1) Assay formats

In addition to, or in alternative to, the detection of Socs2 nucleic acid expression level(s), alterations in expression of Socs2 can be detected and/or quantified by detecting and/or quantifying the amount and/or activity of translated Socs2 polypeptide.

2) Detection of expressed protein

The polypeptide(s) encoded by the Socs2 gene can be detected and quantified by any of a number of methods well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like.

In one preferred embodiment, the Socs2 polypeptide(s) are detected/quantified in an electrophoretic protein separation (e.g. a 1- or 2-dimensional electrophoresis). Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another preferred embodiment, Western blot (immunoblot) analysis is used to detect and quantify the presence of polypeptide(s) of this invention in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the target polypeptide(s).

The antibodies specifically bind to the target polypeptide(s) and may be directly labeled or alternatively may be subsequently detected using labeled antibodies (e.g., labeled sheep anti-mouse antibodies) that specifically bind to a domain of the antibody.

In preferred embodiments, the Socs2 polypeptide(s) are detected using an immunoassay. As used herein, an immunoassay is an assay that utilizes an antibody to specifically bind to the analyte (e.g., the target polypeptide(s)). The immunoassay is thus characterized by detection of specific binding of a polypeptide of this invention to an antibody as opposed to the use of other physical or chemical properties to isolate, target, and quantify the analyte.

Any of a number of well recognized immunological binding assays (see, e.g., U.S. Pat. Nos. 4,366,241; 4,376,110; 4,517,288; and 4,837,168) are well suited to detection or quantification of the polypeptide(s) identified herein.. For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (Socs2 polypeptide). In preferred embodiments, the capture agent is an antibody.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/polypeptide complex.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. ImmunoL, 135: 2589-2542).

Preferred immunoassays for detecting the target polypeptide(s) are either competitive or noncompetitive. Noncompetitive immunoassays are assays in which the amount of captured analyte is directly measured. In one preferred “sandwich” assay, for example, the capture agents (antibodies) can be bound directly to a solid substrate where they are immobilized. These immobilized antibodies then capture the target polypeptide present in the test sample. The target polypeptide thus immobilized is then bound by a labeling agent, such as a second antibody bearing a label.

In competitive assays, the amount of analyte (Socs2 polypeptide) present in the sample is measured indirectly by measuring the amount of an added (exogenous) analyte displaced (or competed away) from a capture agent (antibody) by the analyte present in the sample. In one competitive assay, a known amount of, in this case, labeled polypeptide is added to the sample and the sample is then contacted with a capture agent. The amount of labeled polypeptide bound to the antibody is inversely proportional to the concentration of target polypeptide present in the sample.

In one particularly preferred embodiment, the antibody is immobilized on a solid substrate. The amount of target polypeptide bound to the antibody may be determined either by measuring the amount of target polypeptide present in a polypeptide /antibody complex, or alternatively by measuring the amount of remaining uncomplexed polypeptide.

The immunoassay methods of the present invention include an enzyme immunoassay (EIA) which utilizes, depending on the particular protocol employed, unlabeled or labeled (e.g., enzyme-labeled) derivatives of polyclonal or monoclonal antibodies or antibody fragments or single-chain antibodies that bind Socs2 polypeptide(s), either alone or in combination. In the case where the antibody that binds Socs2 polypeptide is not labeled, a different detectable marker, for example, an enzyme-labeled antibody capable of binding to the monoclonal antibody that binds the Socs2 polypeptide, may be employed. Any of the known modifications of EIA, for example, enzyme-linked immunoabsorbent assay (ELISA), may also be employed. As indicated above, also contemplated by the present invention are immunoblotting immunoassay techniques such as western blotting employing an enzymatic detection system.

The immunoassay methods of the present invention may also be other known immunoassay methods, for example, fluorescent immunoassays using antibody conjugates or antigen conjugates of fluorescent substances such as fluoresceine or rhodamine, latex agglutination with antibody-coated or antigen-coated latex particles, haemagglutination with antibody-coated or antigen-coated red blood corpuscles, and immunoassays employing an avidin-biotin or strepavidin-biotin detection systems, and the like.

The particular parameters employed in the immunoassays of the present invention can vary widely depending on various factors such as the concentration of antigen in the sample, the nature of the sample, the type of immunoassay employed and the like. Optimal conditions can be readily established by those of ordinary skill in the art. In certain embodiments, the amount of antibody that binds Socs2 polypeptides is typically selected to give 50% binding of detectable marker in the absence of sample. If purified antibody is used as the antibody source, the amount of antibody used per assay will generally range from about 1 ng to about 100 ng. Typical assay conditions include a temperature range of about 4° C. to about 45° C., preferably about 25° C. to about 37° C., and most preferably about 25° C., a pH value range of about 5 to 9, preferably about 7, and an ionic strength varying from that of distilled water to that of about 0.2M sodium chloride, preferably about that of 0.15M sodium chloride. Times will vary widely depending upon the nature of the assay, and generally range from about 0.1 minute to about 24 hours. A wide variety of buffers, for example PBS, may be employed, and other reagents such as salt to enhance ionic strength, proteins such as serum albumins, stabilizers, biocides and non-ionic detergents may also be included.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

Antibodies for use in the various immunoassays described herein, are commercially available or can be produced as described below.

3) Antibodies to Socs2 polypeptides

Either polyclonal or monoclonal antibodies may be used in the immunoassays of the invention described herein. Polyclonal antibodies are preferably raised by multiple injections (e.g. subcutaneous or intramuscular injections) of substantially pure polypeptides or antigenic polypeptides into a suitable non-human mammal. The antigenicity of the target peptides can be determined by conventional techniques to determine the magnitude of the antibody response of an animal that has been immunized with the peptide. Generally, the peptides that are used to raise antibodies for use in the methods of this invention should generally be those which induce production of high titers of antibody with relatively high affinity for target polypeptides encoded by Socs2 or variants thereof.

If desired, the immunizing peptide may be coupled to a carrier protein by conjugation using techniques that are well-known in the art. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g. a mouse or a rabbit).

The antibodies are then obtained from blood samples taken from the mammal. The techniques used to develop polyclonal antibodies are known in the art (see, e.g., Methods of Enzymology, “Production of Antisera With Small Doses of Immunogen: Multiple Intradermal Injections”, Langone, et al. eds. (Acad. Press, 1981)). Polyclonal antibodies produced by the animals can be further purified, for example, by binding to and elution from a matrix to which the peptide to which the antibodies were raised is bound. Those of skill in the art will know of various techniques common in the immunology arts for purification and/or concentration of polyclonal antibodies, as well as monoclonal antibodies see, for example, Coligan, et al. (1991) Unit 9, Current Protocols in Immunology, Wiley Interscience).

Preferably, however, the antibodies produced will be monoclonal antibodies (“mAb's”). For preparation of monoclonal antibodies, immunization of a mouse or rat is preferred. The term “antibody” as used in this invention includes intact molecules as well as fragments thereof, such as, Fab and F(ab′)^{2′}, and/or single-chain antibodies (e.g. scFv) which are capable of binding an epitopic determinant. Also, in this context, the term “mab's of the invention” refers to monoclonal antibodies with specificity for a polypeptide encoded by Socs2.

The general method used for production of hybridomas secreting mAbs is well known (Kohler and Milstein (1975) Nature, 256:495). Briefly, as described by Kohler and Milstein the technique comprised isolating lymphocytes from regional draining lymph nodes of five separate cancer patients with either melanoma, teratocarcinoma or cancer of the cervix, glioma or lung, (where samples were obtained from surgical specimens), pooling the cells, and fusing the cells with SHFP-1. Hybridomas were screened for production of antibody which bound to cancer cell lines. Confirmation of specificity among mAb's can be accomplished using relatively routine screening techniques (such as the enzyme-linked immunosorbent assay, or “ELISA”) to determine the elementary reaction pattern of the mAb of interest.

Antibodies fragments, e.g. single chain antibodies (scFv or others), can also be produced/selected using phage display technology. The ability to express antibody fragments on the surface of viruses that infect bacteria (bacteriophage or phage) makes it possible to isolate a single binding antibody fragment, e.g., from a library of greater than 10¹⁰ nonbinding clones. To express antibody fragments on the surface of phage (phage display), an antibody fragment gene is inserted into the gene encoding a phage surface protein (e.g., pIII) and the antibody fragment-pIII fusion protein is displayed on the phage surface (McCafferty et al. (1990) Nature, 348: 552-554; Hoogenboom et al. (1991) Nucleic Acids Res. 19: 4133-4137).

Since the antibody fragments on the surface of the phage are functional, phage bearing antigen binding antibody fragments can be separated from non-binding phage by antigen affinity chromatography (McCafferty et al. (1990) Nature, 348: 552-554). Depending on the affinity of the antibody fragment, enrichment factors of 20 fold -1,000,000 fold are obtained for a single round of affinity selection. By infecting bacteria with the eluted phage, however, more phage can be grown and subjected to another round of selection. In this way, an enrichment of 1000 fold in one round can become 1,000,000 fold in two rounds of selection (McCafferty et al. (1990) Nature, 348: 552-554). Thus even when enrichments are low (Marks et al. (1991) J. Mol. Biol. 222: 581-597), multiple rounds of affinity selection can lead to the isolation of rare phage. Since selection of the phage antibody library on antigen results in enrichment, the majority of clones bind antigen after as few as three to four rounds of selection. Thus only a relatively small number of clones (several hundred) need to be analyzed for binding to antigen.

Human antibodies can be produced without prior immunization by displaying very large and diverse V-gene repertoires on phage (Marks et al. (1991) J. Mol. Biol. 222: 581-597). In one embodiment natural V_{H} and V_{L} repertoires present in human peripheral blood lymphocytes are were isolated from unimmunized donors by PCR. The V-gene repertoires were spliced together at random using PCR to create a scFv gene repertoire which is was cloned into a phage vector to create a library of 30 million phage antibodies (Id.). From this single “naive” phage antibody library, binding antibody fragments have been isolated against more than 17 different antigens, including haptens, polysaccharides and proteins (Marks et al. (1991) J. Mol. Biol. 222: 581-597; Marks et al. (1993). Bio/Technology. 10: 779-783; Griffiths et al. (1993) EMBO J. 12: 725-734; Clackson et al. (1991) Nature. 352: 624-628). Antibodies have been produced against self proteins, including human thyroglobulin, immunoglobulin, tumor necrosis factor and CEA (Griffiths et al. (1993) EMBO J. 12: 725-734). It is also possible to isolate antibodies against cell surface antigens by selecting directly on intact cells. The antibody fragments are highly specific for the antigen used for selection and have affinities in the 1 :M to 100 nM range (Marks et al. (1991) J. Mol. Biol. 222: 581-597; Griffiths et al. (1993) EMBO J. 12: 725-734). Larger phage antibody libraries result in the isolation of more antibodies of higher binding affinity to a greater proportion of antigens.

It will also be recognized that antibodies can be prepared by any of a number of commercial services (e.g., Berkeley antibody laboratories, Bethyl Laboratories, Anawa, Eurogenetec, etc.).

C) Assay optimization

The assays of this invention have immediate utility in screening for agents that modulate the Socs2 expression and/or activity in a cell, tissue or organism. The assays of this invention can be optimized for use in particular contexts, depending, for example, on the source and/or nature of the biological sample and/or the particular test agents, and/or the analytic facilities available. Thus, for example, optimization can involve determining optimal conditions for binding assays, optimum sample processing conditions (e.g. preferred PCR conditions), hybridization conditions that maximize signal to noise, protocols that improve throughput, etc. In addition, assay formats can be selected and/or optimized according to the availability of equipment and/or reagents. Thus, for example, where commercial antibodies or ELISA kits are available it may be desired to assay protein concentration. Conversely, where it is desired to screen for modulators that alter transcription the Socs2 gene, nucleic acid based assays are preferred.

Routine selection and optimization of assay formats is well known to those of ordinary skill in the art.

D) Pre-screening for agents that bind Socs2 or Socs2 polypeptide

In certain embodiments, it is desired to pre-screen test agents for the ability to interact with (e.g. specifically bind to) a Socs2 (or mutant/allele) nucleic acid or polypeptide. Specifically, binding test agents are more likely to interact with and thereby modulate Socs2 expression and/or activity. Thus, in some preferred embodiments, the test agent(s) are pre-screened for binding to Socs2 nucleic acids or to Socs2 proteins before performing the more complex assays described above.

In one embodiment, such pre-screening is accomplished with simple binding assays. Means of assaying for specific binding or the binding affinity of a particular ligand for a nucleic acid or for a protein are well known to those of skill in the art. In preferred binding assays, the Socs2 protein or nucleic acid is immobilized and exposed to a test agent (which can be labeled), or alternatively, the test agent(s) are immobilized and exposed to an Socs2 protein or to a Socs2 nucleic acid (which can be labeled). The immobilized moiety is then washed to remove any unbound material and the bound test agent or bound Socs2 nucleic acid or protein is detected (e.g. by detection of a label attached to the bound molecule). The amount of immobilized label is proportional to the degree of binding between the Socs2 protein or nucleic acid and the test agent.

E) Scoring the assay(s)

The assays of this invention are scored according to standard methods well known to those of skill in the art. The assays of this invention are typically scored as positive where there is a difference between the activity seen with the test agent present or where the test agent has been previously applied, and the (usually negative) control, preferably where the difference is statistically significant (e.g. at greater than 80%, preferably greater than about 90%, more preferably greater than about 98%, and most preferably greater than about 99% confidence level). Most preferred “positive” assays show at least a 1.2 fold, preferably at least a 1.5 fold, more preferably at least a 2 fold, and most preferably at least a 4 fold or even a 10-fold difference from the negative control.

F) Agents for screening: Combinatorial libraries (e.g., small organic molecules)

Virtually any agent can be screened according to the methods of this invention. Such agents include, but are not limited to nucleic acids, proteins, sugars, polysaccharides, glycoproteins, lipids, and small organic molecules. The term “small organic molecules” typically refers to molecules of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

Conventionally, new chemical entities with useful properties are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of potential therapeutic compounds (candidate compounds). Such “combinatorial chemical libraries” are then screened in one or more assays, as described herein to identify those library members (particular chemical species or subclasses) that display a desired characteristic activity. The compounds thus identified can serve as conventional “lead compounds” or can, themselves, be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library such as a polypeptide (e.g., mutein) library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 1233-1250).

Preparation of combinatorial chemical libraries is well known to those of skill in the art. Such combinatorial chemical libraries include, but are not limited to, peptide libraries (see, e.g., U.S. Pat. No. 5,010,175, Furka (1991) Int. J Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, Dec. 26, 1991), encoded peptides (PCT Publication WO 93/20242, Oct. 14, 1993), random bio-oligomers (PCT Publication WO 92/00091, Jan. 9, 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines 5,288,514, and the like).

Devices for the preparation of combinatorial libraries are commercially available (see, e.g., 357 MPS, 390 MPS, Advanced Chem Tech, Louisville, Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include, but are not limited to, automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist and the Venture™ platform, an ultra-high-throughput synthesizer that can run between 576 and 9,600 simultaneous reactions from start to finish (see Advanced ChemTech, Inc. Louisville, Ky.)). Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

G) High Throughput Screening

Any of the assays for compounds modulating the accumulation or degradation of metabolic products described herein are amenable to high throughput screening. Preferred assays detect increases or decreases in Socs2 transcription and/or translation in response to the presence of a test compound.

The cells utilized in the methods of this invention need not be contacted with a single test agent at a time. To the contrary, to facilitate high-throughput screening, a single cell may be contacted by at least two, preferably by at least 5, more preferably by at least 10, and most preferably by at least 20 test compounds. If the cell scores positive, it can be subsequently tested with a subset of the test agents until the agents having the activity are identified.

High throughput assays for various reporter gene products are well known to those of skill in the art. For example, multi-well fluorimeters are commercially available (e.g., from Perkin-Elmer).

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

H) Modulator databases

In certain embodiments, the agents that score positively in the assays described herein (e.g. show an ability to modulate Socs2 expression) can be entered into a database of putative and/or actual modulators of Socs2 expression. The term database refers to a means for recording and retrieving information. In preferred embodiments, the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

III. Pre-screening for agents that bind Socs2 or Socs2 polypeptide

In certain embodiments it is desired to pre-screen test agents for the ability to interact with (e.g. specifically bind to) a hg gene (or mutant/allele) nucleic acid or polypeptide. Specifically, binding test agents are more likely to interact with and thereby modulate Socs2 expression and/or activity. Thus, in some preferred embodiments, the test agent(s) are pre-screened for binding to Socs2 nucleic acids or to Socs2 proteins before performing the more complex assays described above.

In one embodiment, such pre-screening is accomplished with simple binding assays. Means of assaying for specific binding or the binding affinity of a particular ligand for a nucleic acid or for a protein are well known to those of skill in the art. In preferred binding assays, the Socs2 protein or nucleic acid is immobilized and exposed to a test agent (which can be labeled), or alternatively, the test agent(s) are immobilized and exposed to an Socs2 protein or to a Socs2 nucleic acid (which can be labeled). The immobilized moiety is then washed to remove any unbound material and the bound test agent or bound Socs2 nucleic acid or protein is detected (e.g. by detection of a label attached to the bound molecule). The amount of immobilized label is proportional to the degree of binding between the Socs2 protein or nucleic acid and the test agent.

IV. Screening for hg phenotype or for a predilection thereto

In still another embodiment, this invention contemplates screening an organism for the presence of an hg phenotype (e.g. a Socs2 knockout or Socs2 dysregulation). In preferred embodiments, such methods involve either detecting a Socs2^{hg} mutation or other “downregulated” Socs2 allele or mutant or modified nucleic acid or a protein product of such a nucleic acid. Nucleic acids and/or proteins can be detected by a wide variety of methods well known to those of skill in the art, e.g as described above.

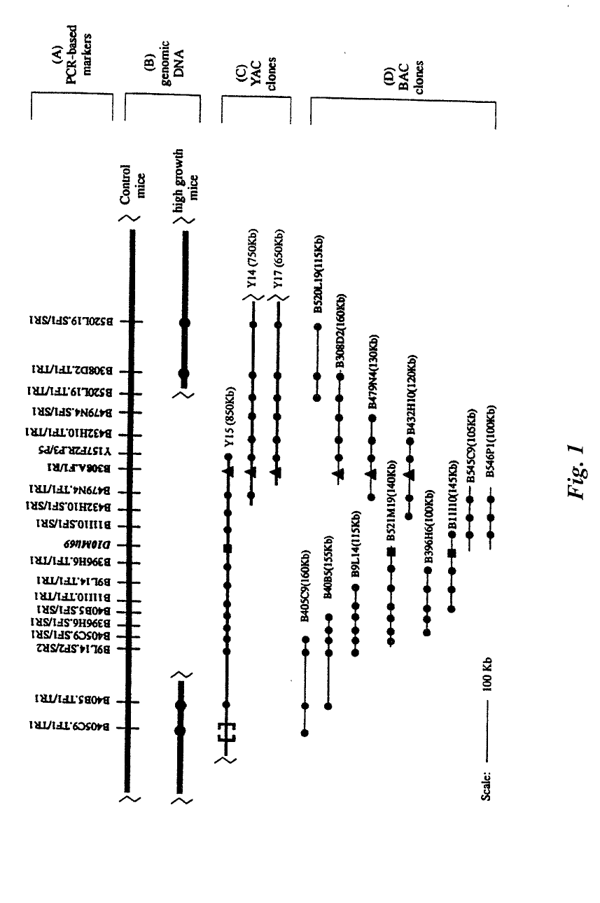

Thus, an aspect of this invention is to use oligonucleotide probes to detect DNA sequences complementary to the probes e.g. in a mixture of DNA sequences (genomic DNA, mRNA, etc.). Or to select oligonucleotide primers for amplifying such nucleic acid sequences. Certain Preferred primers comprise at least 10, preferably at least 15, more preferably at least 20, and most preferably at least 25, or at least 30 contiguous nucleotides, e.g., from SEQ ID NOS:1, 3, 9, etc or their complementary sequences.. For example, among the PCR primers that are markers of the hg region and that have been used to amplify the STSs shown in the physical map of FIG. 1 are the following single stranded oligonucleotide sequences:

TGGAAGCCAGAGACAAGCAG SEQ ID NO:5

AGAAATGGAAGCCAGAGACAA SEQ ID NO:6

CTTTTGACACCTTCCTCGATTC SEQ ID NO:7

CTCAAACCACAGGCCTCCGGA SEQ ID NO:8

V. Hg nucleic acids and vectors

In certain embodiments, practice of this invention includes the use of a nucleic acid construct comprising a sequence coding for hg. In certain embodiments, the hg nucleic acid construct is present in an expression cassette. A “recombinant expression cassette” or simply an “expression cassette” is a nucleic acid construct, generated recombinantly or synthetically, with nucleic acid elements that are capable of effecting expression of a gene or cDNA in hosts compatible with such sequences. Expression cassettes typically include at least promoters and optionally, transcription termination signals. Typically, the recombinant expression cassette includes a nucleic acid to be transcribed (e.g., a nucleic acid encoding a desired polypeptide), and a promoter. Additional factors necessary or helpful in effecting expression may also be used as described herein. For example, an expression cassette can also include nucleotide sequences that encode a signal sequence that directs secretion of an expressed protein from the host cell a selectable marker, and the like.

The expression cassette is, optionally, placed in a vector (e.g., a bacteria, insect, or viral vector for transfecting one or more cells. Expression and cloning vectors preferably contain a nucleotide sequence that enables the vector to replicate in one or more selected host cells. Generally, in cloning vectors this sequence is one that enables the vector to replicate independently of the host chromosomes, and includes origins of replication or autonomously replicating sequences.

Methods of expressing heterologous proteins are well known to those of skill in the art and using, the information provided herein one of skill in the art can routinely prepare appropriate expression cassettes, vectors, transformed cells, and the like. Examples of these techniques and instructions sufficient to direct persons of skill through many cloning exercises are found in Berger and Kimmel, Guide to Molecular Cloning Techniques, Methods in Enzymology 152 Academic Press, Inc., San Diego, Calif. (Berger); Sambrook et al. (1989) Molecular Cloning—A Laboratory Manual (2nd ed.) Vol. 1-3, Cold Spring Harbor Laboratory, Cold Spring Harbor Press, NY, (Sambrook et al.); Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Cashion et al., U.S. Pat. No. 5,017,478; and Carr, European Patent No. 0,246,864. Examples of techniques sufficient to direct persons of skill through in vitro amplification methods are found in Berger, Sambrook, and Ausubel, as well as Mullis et al., (1987) U.S. Pat. No. 4,683,202; PCR Protocols A Guide to Methods and Applications (Innis et al. eds) Academic Press Inc. San Diego, Calif. (1990) (Innis); Ainheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad Sci. USA 87, 1874; Lomell et al. (1989) J. Clin. Chem., 35: 1826; Landegren et al., (1988) Science, 241: 1077-1080; Van Brunt (1990) Biotechnology, 8: 291-294; Wu and Wallace, (1989) Gene, 4: 560; and Barringer et al. (1990) Gene, 89: 117.

VI. Kits

In still another embodiment, this invention provides kits for creation of animals comprising an inhibited or knocked-out Socs2 gene and/or for screening for Socs2 expression. Kits for the preparation of knockout animals preferably include a nucleic acid that, upon undergoing homologous recombination with a Socs2 gene or Socs2 control element inhibits or eliminates transcription and/or translation of a Socs2 gene product. Such kits, optionally, include reagents for delivery of such nucleic acids and appropriate instructional materials.

Other kits comprises cells (e.g. stem cells), cell lines, embryos, or animals comprising a cell encoding a Socs2 allele or alleles that results in reduced or eliminated expression of Socs2. Where the kits comprise animals, the animals are heterozygous or homozygous for the “mutant” socs2 allele(s). The “mutation” can exist in somatic an/or reproductive cells and, in certain embodiments, the animal is chimeric for the “mutation.”

In still other embodiments, this invention provides kits for performing one or more of the assays described herein. Preferred kits comprise one or more nucleic acid probes specific to Socs2 or to a mutant thereof and/or one or more antibodies specific to a Socs2 polypeptide. Also, optionally included, are buffers, equipment (e.g. microtiter plates) and the like to facilitate practice of the assays described herein.

As indicated above, the kits may include instructional materials containing directions (i.e., protocols) for the preparation of Socs2 knockouts, and/or for the practice of the assay methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

SEQ ID NO:1 illustrates a mouse cDNA, which is a gene in the high growth (“hg”) region. The hg region appears to be highly conserved, as will be more fully described herein.

Turning to FIG. 1, the deleted microsatellite marker, D10Mit69, was utilized as an entry point to physical cloning of the hg-containing segment using Yeast Artificial Chromosome (YAC) and Bacterial Artificial Chromosome (BAC) cloning systems. The size of the deletion in high growth mice, estimated from the clone lengths, is on the order of a half million base pairs.

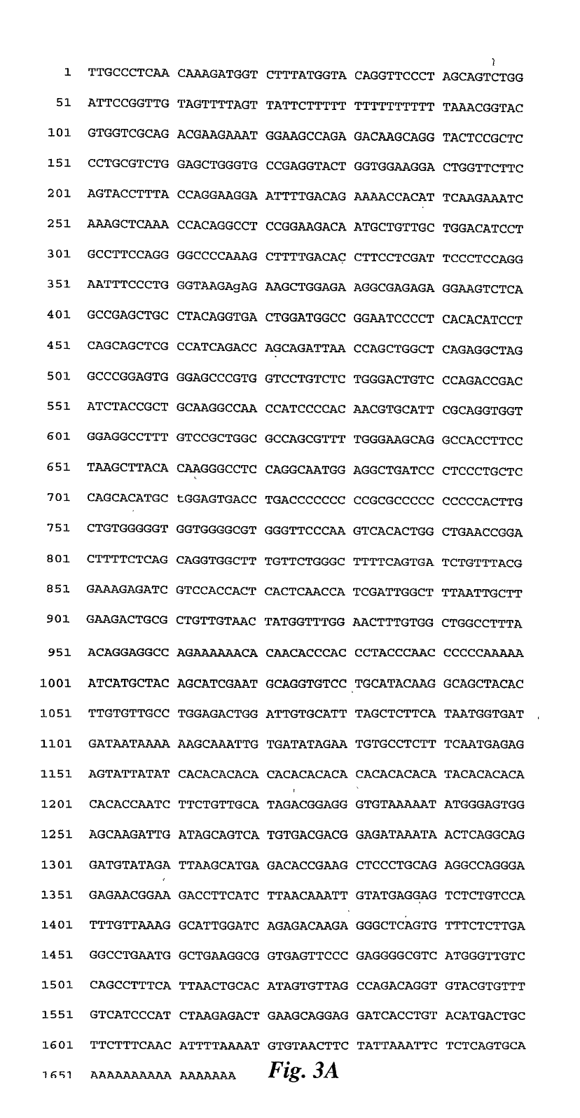

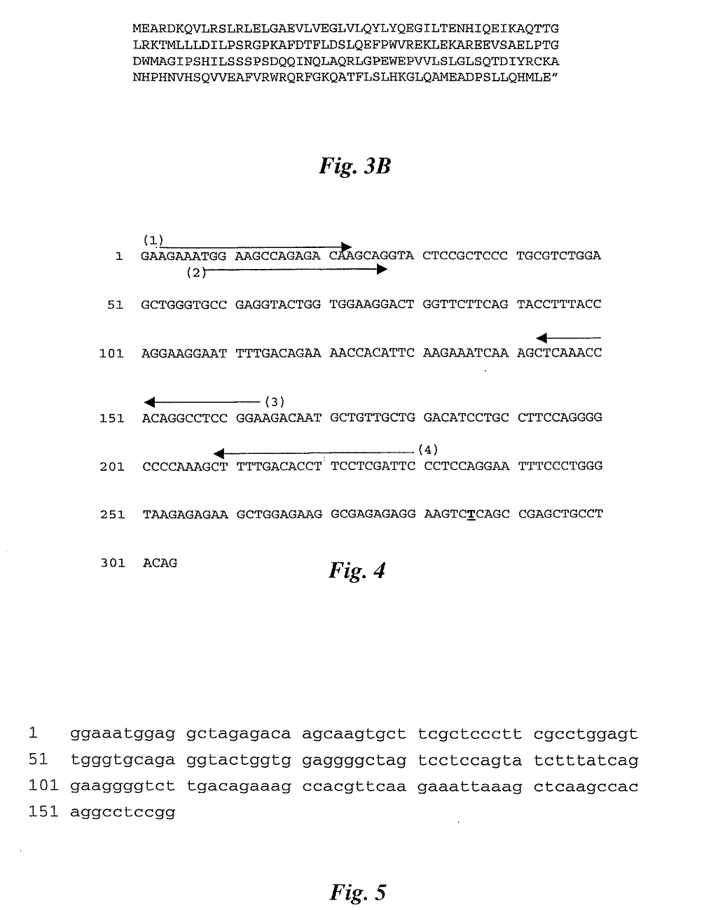

The open reading frame of the mouse B308A-6-1 (FIG. 3A) is predicted to encode 199 amino acids (FIG. 3B) which share very high homology (178/199 identical amino acids) with the human protein, corresponding to the RAIDD/CRADD gene. The nucleotide sequence of the original exon-trap clone, with the position of primers 1, 2, 3, and 4 is indicated by FIG. 4 (this partial sequence is SEQ ID NO:2). The bovine sequence, which is yet a partial coding sequence, is SEQ ID NO:3 and is shown by FIG. 5. This PCR amplified sequence was from reverse transcribed lactating mammary gland mRNA using the mouse primers 2 and 3 (the primers indicated by FIG. 4).

There is high homology observed between the mouse and bovine sequences (49/52 identical amino acids). The homology between the mouse protein discussed here and the human RAIDD proteins reported by another group (Duan and Dixit (1997) Nature 385:86-89) is very conserved when compared in the conserved, NH2 and C terminal domains. In the NH₂-terminal domain (amino acids 1-80), 79 out of 80 residues are identical, whereas in the C-terminal “death domain” (amino acids 123-199), 77 out of 86 amino acids are identical. Therefore PCR primers in these domains should be very useful to pick up the homologous DNA segments in other species. The bovine B308A segment, SEQ ID NO:3, was obtained using mouse primers on the NH₂-terminal domain.

Without being bound by theory, we suggest that high growth mice are bigger because they have more cells that are moderately larger. The control of cell number depends primarily on the balance between processes of proliferation and cell death (apoptosis) (Jacobson et al. (1997) Cell, 88: 347-354, 1997; Raff (1996) Cell, 86:173-175). In mammals, apoptosis begins at blastula stage and continues throughout life and can be of equal importance in controlling cell numbers as cell proliferation (Raff, supra). There are nematode mutations that abolish apoptosis and result in a worm with a 15% increase in cell numbers, a normal lifespan, morphology and behavior (Ellis and Horvitz (1986) Cell, 44: 817-829). It might be possible therefore that the high growth phenotype as a result of an increase in cell numbers might be due to in part to a perturbed apoptosis program caused by a lack of function of a apoptotic protein such as that corresponding to our clone B308A-6-1, which is homologous to human RAIDD (Duan and Dixit, supra).

Returning to FIG. 1, we have cloned a high growth (“hg”) region in bacterial artificial chromosomes (“BAC”) and yeast artificial chromosomes (“YAC”). Marker D10Mit69 was used to initiate the bi-directional chromosomal walk. A BAC library (Research Genetics, Huntsville, Ala., USA) was screened as follows: so-called higher-order pools containing DNAs from several 384 clone plates were screened by polymerase chain reaction (PCR) to identify a positive 384 well plate. Clones from this plate were then grown on membranes, colony-lysed (Nizetic et al. (1991) Proc. Natl. Acad. Sci., USA, 88(8):3233-3237, 1991) and hybridized to a relevant probe. If a probe was a microsatellite marker (such as D10Mit69) or contained other types of repetitive DNA, an oligonucleotide probe was designed in the unique parts of the marker to prevent cross hybridization to clones containing these repeats. Identified single positive BAC clones were sized on a pulsed-field gel apparatus (CHEF-DRIII, Bio-Rad) and sequenced from the ends of the insert (Wang et al. (1994) Genomics, 24:527-534). These sequences were utilized to construct a PCR primer pair at each end (so-called sequence tagged sites, “STS,” which were in turn used to screen the BAC library again to isolate the next overlapping clone(s). Each end STS was examined for amplification in hg mouse and its parental strains to test whether a deletion breakpoint had been crossed. The cloning of the hg region in BAC clones was complete once clones that span the whole deletion and both deletion endpoints were obtained. A map of YAC and BAC clones in the hg region is illustrated by FIG. 1.

BAC clones B308D2 and B11I10 (FIG. 1) were subcloned in vector pSPL3 (GibcoBRL, New York, USA) which flanks an insert with splice donor and splice acceptor sites. These pSPL3 subclones of BACs were transfected into COS7 cells (African-green monkey cells obtained from American type culture Collection, Maryland, USA) using electroporation following manufacturer's protocols (BioPulser, Bio-Rad, California, USA). RNA was isolated from cell cultures 24 hours following the transfection using Trizol reagent (Gibco-BRL). Reverse transcription and PCR amplification were as described in Church et al. (1994) Nat. Genet., 6: 98: 105. Each exon trapping product was then cloned (TA cloning kit, Invitrogen, USA) and used as a probe in hybridizations to blots of BAC digests to verify whether the exon trapping product was derived from a particular BAC(s). Candidate exon trapping products were then sequenced.

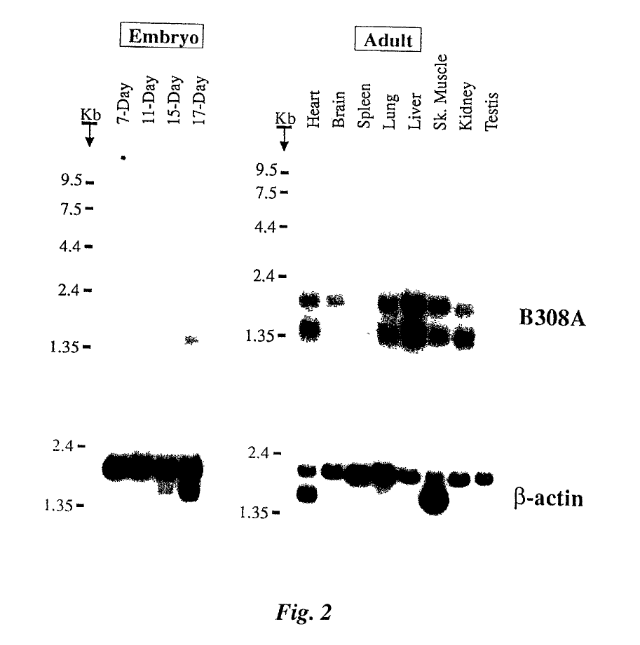

The sequence of end STSs and candidate exon trapping products were compared to all sequences in public sequence data banks with BLAST and FASTA computer programs to test for similarity to known genes or expressed sequence tags (ESTs). Candidate exons were then screened for the presence of corresponding RNA from a variety of tissues and developmental stages using Northern blots. The sequence of the candidate exon trapping product B308A was found to be highly homologous to an EST derived from the mouse embryo (93% identity) and several human ESTs (83-87% identity) derived from fetal liver/spleen and infant brain, and to the human death adaptor molecule (86% similarity), RAIDD (GenBank Accession No. 079115) (Duan and Dixit, supra) or CRADD (GenBank Accession No. 084388) (Ahmad et al. (1997) Cancer Research, 57: 615-617). The Northern analysis showed that the RNA containing exon trapping B308A sequence is widely expressed in several tissues and developmental stages, most notably in liver (FIG. 2).

Using the candidate exon B308A as a probe, a mouse mammary (15-day gestation) cDNA library (C. Watson, Roslin Institute, personal communication) was screened using standard procedures for lambda phage cDNA library screens (Maniatis et al. (1992) Molecular Cloning: A Laboratory Manual, Cold Spring Harbor Lab., Cold Spring Harbor, New York). A total of 11 lambda phage cDNA clones were isolated—one clone of ˜1.6 Kb and 10 clones of ˜1 Kb. The ˜1.6 Kb clone (clone B308A-6-1, FIG. 3) was then thoroughly sequenced using primer walking method. B308A-6-1 was also mapped back to the hg deletion (FIG. 1). The cDNA containing B308A-6-1, SEQ ID NO:1, represents the first candidate gene in the hg region..

Using mouse PCR primers we have amplified and sequenced a fragment corresponding to the NH₂-terminal domain of B308A-6-1 in reverse transcribed RNA from cow lactating mammary gland (FIG. 5). A comparison between the predicted amino acid sequences of mouse, bovine, and human proteins shows that the sequence of B308A-6-1 is highly conserved between mouse, human, and cattle. This lends experimental support to the conservation of this region in other animals and suggests hg will most likely be found in the genome of other domestic animals, including poultry.

The mouse cDNA, SEQ ID NO:1, is used isolate a cosmid containing chicken hg.. The hg homolog was checked by DNA sequencing and the gene mapped by fluorescent in-situ hybridization (“FISH”) onto chicken metaphase spreads to chicken chromosome 1 (Smith et al. Mamm Genome 11:706-709, 2000). Markers were developed based on the chicken genomic sequence and mapped onto a reference genetic linkage map of chicken (the map being available at the Web site http://www.ri.bbsrc.ac.uk/). Interestingly Raidd/Cradd maps to the approximate location of a growth QTL in broiler chickens (Groenen et al. (1997) Anim. Biotechnology 8:41-46). The hg marker can be used to genotype F₂ progeny of the cross to confirm linkage to the growth QTL.

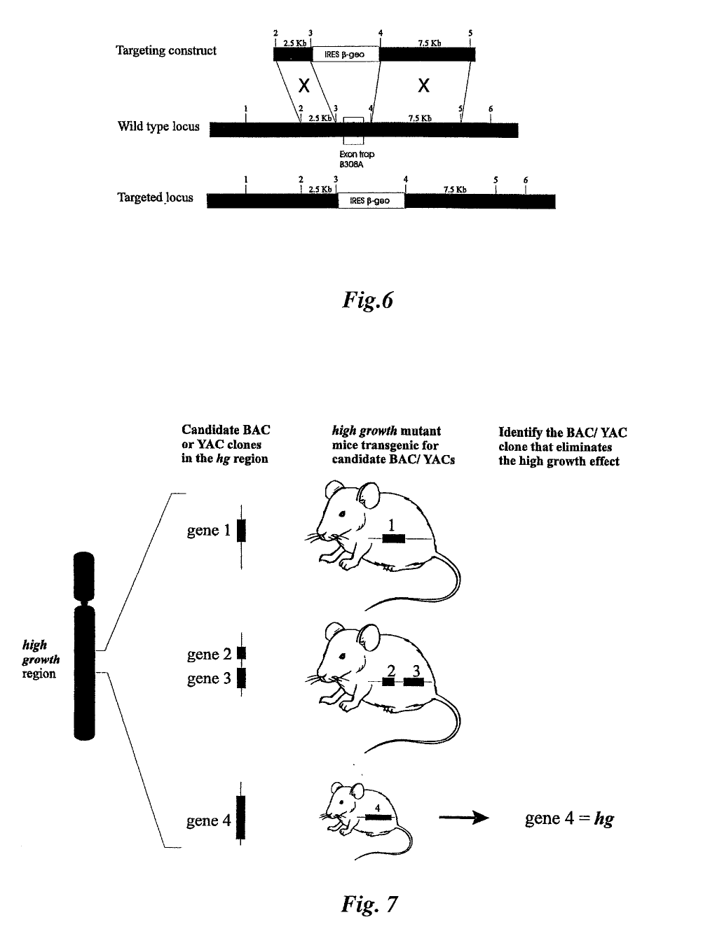

Since in mammals there is a gene about every 50 kb and a deletion in high growth mice is about 500 Kb, further transcript mapping may identify additional genes in the hg region (see, e.g., SEQ ID NO: 20). The deletion in the hg region suggests that high growth effect is due to a lack-of-function of hg. Therefore any gene that maps to the deletion may contribute to the hg phenotype. A transgenic analysis for gene identification is conducted as shown in FIGS. 6 and 7.

It is feasible to identify cognate genes by in vivo complementation. The addition of wild type copies of the hg gene onto a high growth mutant background is expected to eliminate the high growth effect. High growth mice transgenic for hg are expected to grow more slowly than non-transgenic high growth mice. For this purpose, transgenic mice containing candidate DNA constructs or candidate large insert clones such as YACs and BACs are constructed. Transgenic lines carrying these constructs are then be tested for their ability to complement the hg mutation in breeding studies. We have created 2 transgenic lines containing BAC clones B11I10 and 520L19 (contains the Socs2 gene) that are being used to study the effects of in-vivo complementation of homozygous hg/hg mice, and to study the effects of overexpression of genes like Socs2 on wild type +/+ mice.

### Example 2

### Lack of Socs2 Expression Causes the High-Growth Phenotype in Mice

Characterizing causal molecular defects in mouse models of overgrowth or dwarfism helps to identify the key genes and pathways that regulate the growth process. We report here the molecular basis for high growth (hg), a spontaneous mutation that causes a 30-50% increase in postnatal growth. We conclude that hg is an allele of the suppressor of cytokine signaling 2 (Socs2), a member of a family of regulators of cytokine signal transduction. We demonstrate mapping of Socs2 to the hg region, lack of Socs2 mRNA expressin, a disruption of the Socs2 locus in high growth (HG) mice and a similarity of phenotypes of HG mice and Socs2^{-/-} mice generated by gene targeting. Charactersitics of the HG phenotype suggest that Socs2 deficiency affects growth prenatally and postnatally, most likely through deregulating the growth hormone (GH)/insulin-like growth factor I (IGF1). These results demonstrate a critical role for Socs2 in controlling growth.

The study of mammalian growth-control genes is essential for elucidating the mechanism of growth at the tissue, organ or whole-body level. The high growth (hg) mutation is a unique overgrowth model in that it causes a 30-50% increase in postweaning growth without resulting in obesity (Bradford and Famula (1984) Genet. Res. 44: 293-308). High growth (HG) mice have increased plasma IGF1 (Corva and Medrano (2000) Physiol. Genomics 3: 17-23; Medrano et al. (1991) Genet. Res. 58: 67-74) and decreased plasma and pituitary GH (Medrano et al. (1991) Genet. Res. 58: 67-74) suggesting that the causal mutation influences growth through deregulating the GH/IGF1 system. We have shown (Horvat and Medrano (1995) Genetics 139: 1737-1748) that hg is not an allele of Gh or Igfl and that it is located within a 500-kb deletion in mouse chromosome 10 (Horvat and Medrano (1998) Genomics 54: 159-164). Here we used comparative mapping to identify positional candidates for hg. The mouse hg region was previously mapped (Horvat and Medrano (1998) Genomics 54: 159-164) to a genetic interval of 100 to 103 cM from the top of human chromosome 12. Human expressed sequence (EST) clones from this region were selected from the human gene map (http://www.ncbi.nlm.nih.gov/) and used as probes on blots containing mouse BAC clones from the hg region. Southern analysis was performed using standard procedures (Sambrook et al.(1989) Molecular cloning: a laboratory manual. 2nd ed., Cold Spring Harbor Laboratory Press, Cold Spring Harbor, NY.) under the lower stringency hybridization temperature (55° C.).

One human EST clone (IMAGE ID 133063) representing a cDNA for the human suppressor of cytokine signaling (SOCS2) cross-hybridized to BAC 520L19 that was previously mapped at the deletion endpoint in the hg region (Horvat and Medrano (1998) Genomics 54: 159-164). Comparative sequence analyses established that mouse, human and rat SOCS2 cDNAs (Unigene clusters Mm 4132, Mm. 30754, Hs. 110776 and Rn. 15045) are homologous to a sequence from the BAC 520L19 (FIG. 2a). SOCS2 protein, also known as cytokine-inducible SH2-containing protein 2 (CISH2), belongs to a family of cytokine-inducible inhibitors that regulate cytokine signal transduction (Starr et al. (1997) Nature 387: 917-921). SOCS2 is involved in GH signaling (Favre et al. (1999) FEBS Letters 453: 63-66) and was shown to interact with the IGF1 receptor (Dey et al. (1998) J. Biol Chem. 273: 24095-24101) linking SOCS2 to two key molecules in mammalian growth control. Therefore, mapping of Socs2 to the hg region and its potential function in growth control via GH/IGFI made Socs2 an excellent candidate for hg.

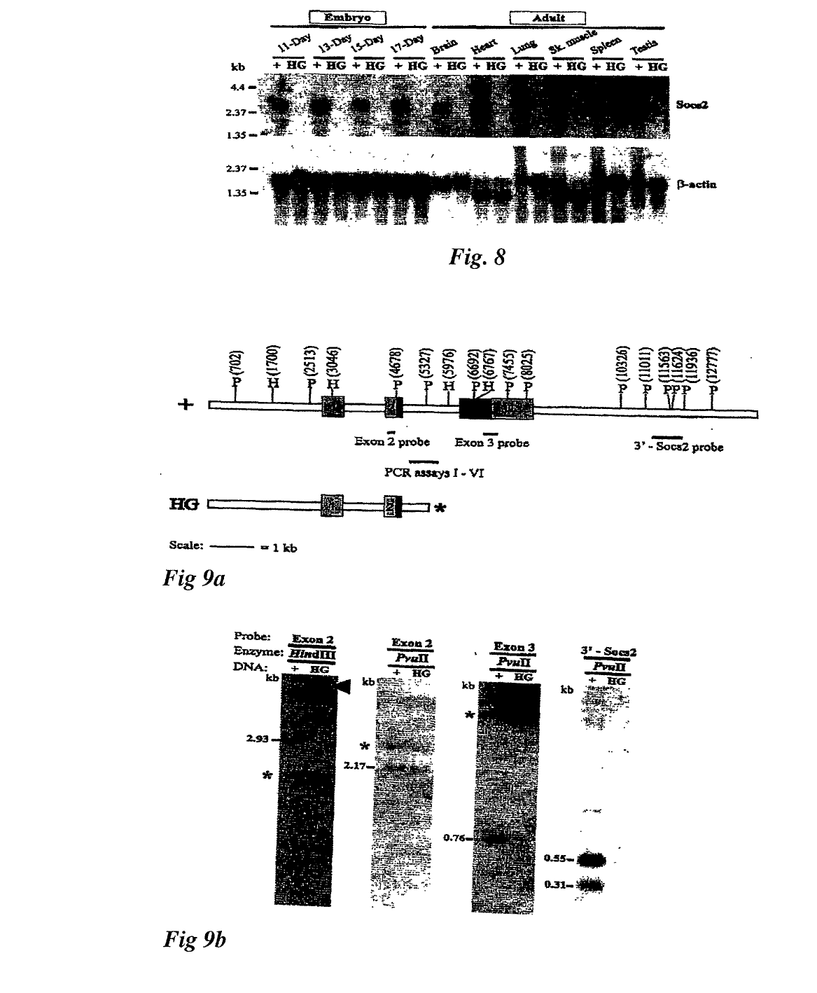

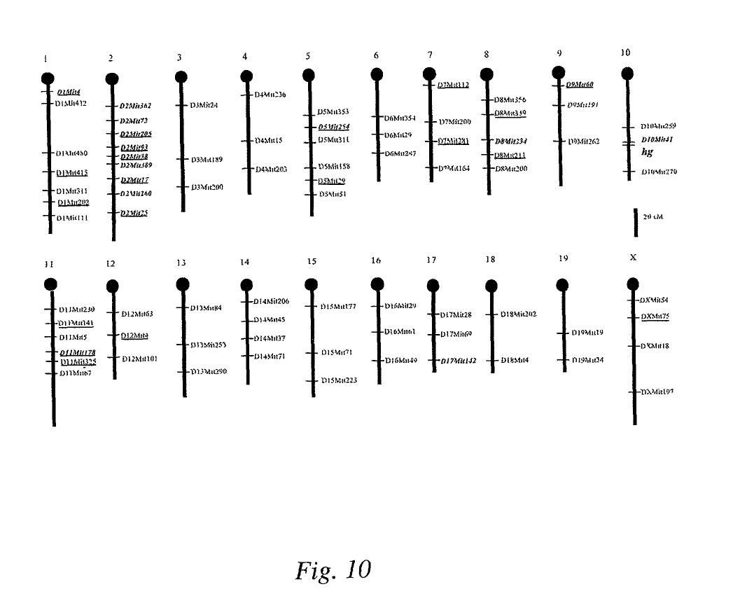

We examined whether Socs2 expression is affected in the HG model and found a lack of Socs2 mRNA in HG mice (FIG. 8). To identify an underlying cause for this, we searched for genomic alterations at the Socs2 locus in HG mice. Southern, polymerase chain reaction (PCR) and sequence analyses demonstrated a deletion breakpoint in HG mice in intron 2 of Socs2 (FIG. 9), which results in the complete loss of exon 3 and sequences downstream. We note that the phenotype of Socs2-deficient mice (Socs2^{-/-}) generated by gene targeting (Metcalf et al. (2000) Nature 405: 1069-1073) is essentially identical to the phenotype of HG mice. Similar to Socs2^{-/-} mice, the postweaning growth of HG mice is increased by 30-50% (Bradford and Famula (1984) Genet. Res. 44: 293-308), collagen content in skin is increased (Reiser et al. (1996) Am. J Physiol. 271: 8696-703), organ and skeletal growth are increased (Famula et al. (1988) Growth. Dev. & Aging 52: 145-150), the increase in muscle mass is accompanied by muscle fiber hyperplasia (Summers and Medrano (1994) Growth Dev. Aging 58: 135-148, Summers and Medrano (1997) Proc. Soc. Exp. Biol. Med. 214: 380-385) and GH secretion and IGF1 secretion are deregulated (Corva and Medrano (2000) Physiol. Genomics 3: 17-23; Medrano et al. (1991) Genet. Res. 58: 67-74). On the basis of mapping of Socs2 to the hg region, the similarity of phenotypes between Socs2^{-/-} and HG mice, and the lack of Socs2 expression and disruption of the Socs2 locus in HG mice described above, we conclude that hg is an allele of Socs2 (Socs2^{hg}) and that the lack of Socs2 expression is responsible for the high growth phenotype.

A potential dissimilarity between the Socs2^{-/-} mice and HG mice is in serum IGF1 levels. No differences were reported between Socs2^{-/-} and control mice (Metcalf et al. (2000) Nature 405: 1069-1073) whereas we have demonstrated elevated serum IGF1 levels in HG mice (Corva and Medrano (2000) Physiol. Genomics 3: 17-23; Medrano et al. (1991) Genet. Res. 58: 67-74). In Socs2^{-/-} mice, it was suggested that Socs2-deficiency leads to increased local production of IGF1, which is then responsible for the increased growth phenotype (Metcalf et al. (2000) Nature 405: 1069-1073). Despite the recent evidence that paracrine/autocrine action of IGF1 is of paramount importance in regulating postnatal growth (Sjogren et al. (1999) Proc. Natl. Acad. Sci, USA, 96: 7088-7092; Yakar et al. (1999) Proc. Natl. Acad. Sci., USA, 96: 7324-7329), the fact that we find increased serum levels of IGF1 in HG mice can not rule out at least some endocrine role of IGF1 in generating the increased growth phenotype. Given that the genetic background (strain C57BL/6J) is the same in both Socs2^{-/-} and HG mice, it is possible that the discrepancy is due to differences in the age of mice when IGF1 serum levels were determined or husbandry procedures. Another possible explanation could be that the increased IGF1 serum level in HG mice is not due to Socs2 deficiency but due to another effect of the hg region (e.g., deletion of another gene within the 500 kb deletion).

We also show that the Socs2 mRNA is expressed during embryogenesis in wild type mice (FIG. 8) suggesting its role in prenatal growth. It is possible that Socs2 deficiency has an effect during fetal growth, weeks before the effect on body weight is observed and before the major GH/IGF1 effect on postnatal growth (about 3-4 weeks of age) starts. Evidence that embryogenesis is affected in the HG mouse is in the fetal muscle, where delayed myogenesis and muscle fiber hyperplasia were observed (Summers and Medrano (1997) Proc. Soc. Exp. Biol. Med. 214: 380-385). Muscle fiber hyperplasia has also been suggested to occur in Socs2^{-/-} mice (Metcalf et al. (2000) Nature 405: 1069-1073). Therefore, Socs2 deficiency has an effect on fetal muscle development resulting in more muscle cells and might have a similar effect in other fetal tissues. The prenatal effect of Socs2 deficiency could be mediated via GH and IGF1, which have been implicated in fetal growth and development (Shoba et al. (1999) Mol. Cell. Endo. 152: 125-136). Therefore, the observed effect on postnatal growth may not entirely be due to the postnatal effect of Socs2 deficiency but also due to the prenatal effect of Socs2 deficiency mediated through changes of developmental program of some fetal tissues.

Increased growth in HG mice and identified genomic and expression alterations of Socs2 confirm the key role for this gene in growth control. The phenotypes of Socs2^{-/-} mice and HG mice are very similar in several characteristics (see above), but a question still remains whether the HG mice have some additional phenotypes caused by the 500 kb deletion. Further comparisons of growth and other traits between the Socs2^{-/-} mice, HG mice and knockouts of other genes from the hg region (e.g., Raidd/Cradd) should help to clarify if there are any other phenotypes particular to the HG mice that are not due to Socs2 deficiency alone.

For a growth-control gene with an inhibitory function like Socs2, it is expected that the lack of expression has a growth promoting effect, and overexpression has a growthinhibiting effect (Efstratiadis (1998) Int. J Dev. Biol. 42: 955-976). Therefore, manipulation of SOCS2 protein expression should be useful in animals as a strategy for improving animal growth and in human medicine for treating growth disorders.

### Example 3

### Quantitative Trait Loci Affecting Growth in High Growth (hg) Mice

This example describes a genome-wide scan performed in order to identify Quantitative Trait Loci (QTL) associated with growth in a population segregating high growth (hg), a partially recessive mutation that enhances growth rate and body size in the mouse. A sample of 262 hg/hg mice was selected from a C57BL/6J-hg/hg×CAST/EiJF₂ cross and typed with 79 SSLP markers distributed across the genome. Eight significant loci were identified through interval mapping. Loci on chromosomes 2 and 8 affected the growth rate of F2 mice. Loci on chromosomes 2 and 11 affected growth rate ad carcass lean mass(protein and ash). A locus on chromosome 9 modified femur length and another one in chromosome 17 affected both carcass lean mass and femur length, but none of these had significant effects on growth rate. Loci on chrosomes 5 and 9 modified carcass fat content. Additive effects were positive for C57BL/6J alleles except for the two loci affecting carcass fatness. Typing of selected markers in 274 +/+ F2 mice revealed significant interactions between hg and other QTL, which were detected as changes in gene action (additive or dominant) and in allele substitution effects. Knowledge about interactions between loci, especially when major genes are involved, helps in the identification of positive candidate genes and in understanding of the complex genetic regulation of growth rate and body size in mammals.

### Introduction

The high growth locus (hg) is an autosomal, partially recessive mutation that enhances weight gain and body size by 30-50% in the mouse (Bradford and Famula (1984) Genet. Res. 44: 293-308). Despite the drastic change in growth rate, hg/hg mice are proportionate in the size of tissues and organs (Famula et al. (1988) Growth Dev. Aging 52: 145-150). This unique phenotype distinguishes hg from other known spontaneous mutations that affect body weight by causing either obesity (LEP^{ob}, Lepr^{db}, Cpe^{fat}, A^{y}, tub) or dwarfism (Pitl^{dw}, Propl^{df}, Ghrhr^{lit}, Hmgic^{pg}, mn, dm) (Lyon et al. (1996) Genetic Variants and Strains of the Laboratory Mouse. Oxford University Press, Oxford; New York). Genetic and physical mapping have determined that a deletion in chromosome 10 causes the High Growth (HG) phenotype (see discussion herein and Horvat and Medrano (1996) Genomics 36: 546-549; Horvat and Medrano (1998) Genomics 54: 159-164). It was a discovery of the present invention that the hg phenotype results from a lack of expression of the suppressor of cytokine signaling 2 (Socs2 or Cish2).

A spontaneous mutation enhancing growth rate and body size is a valuable model for studying the genetics of growth in mammals. Changes in body size are usually achieved through an altered pattern of cell proliferation (Raff (1996) Cell 86: 173-175). It has been demonstrated that, at least in the muscle, hg mice have a larger number of fibers due to enhanced cell proliferation and delayed fusion of myoblasts (Summers and Medrano (1994) Growth Dev. Aging 58: 135-148; Summers and Medrano (1997) Proc. Soc. Exp. Biol. Med. 214: 380-385). However, there is no evidence of any abnormalities in tissue development as it is observed in other mouse models of enhanced growth, such as the p27^{Kipl} gene knockout (Nakayama et al. (1996) Cell 85: 707-720).

One of the potential limitations to the extension to other species of discoveries from major gene mutations in the mouse is the confounding effect of other genetic and non-genetic factors on the phenotype under study, and hg is not an excepton to that limitation. We have demonstrated that the nutritional environment has a profoud effect on the hg phenotype (Corva and Medrano (2000) Genomics 3: 17-23). Also, other lines of evidence suggest that the genetic background could modulate the effects of hg on growth. The hg locus was found in a strain of mice selected for high 3- to 6-week weight gain (Bradford and Famula (1984) Genet. Res. 44: 293-308). In that genetic background, weight gain data followed a bimodal distribution and it was possible to identify most hg/hg mice based on their phenotype. A C57BL/6J-hg/hg×CAST/EiJ F₂ cross (Horvat and Medrano (1995) Genetics 139: 1737-1748, and examples herein) was used to map hg to chromosome 10, and in this population, weight gain data followed a normal distribution and it was no longer possible to identify hg/hg individuals without knowledge of their genotypes. This information, together with the fact that hg was discovered in aline selected for high weight gain, led us to hypothesize that expressivity of hg was modulated by other genes associated with growth regulation.

In this example, we present the results of a genomo-wide scan on the hg/hg individuals of a C57BL/6J-hg/hg×CAST/EiJ F₂ cross to identify QTL (Quatitative Trait Loci) affecting growth rate, body size, and carcass composition. After identifying QTL in the hg/hg individuals we examined the effect of these QTL also in +/+ F₂ mice, for identifying genetic modifier loci of hg. We considered as unique modifiers of hg all the QTL that were detected in the hg/hg background, but not in the +/+ background and those that had effects in both backgrounds, but displayed significant differences in gene action.

### Materials and Methods

### Mouse Crosses

The hg locus has been introgressed into the C57BL/6J (C57) background by nine backcrosses to create the congenic line C57BL/6J-hg/hg (HG). In this experiment, congenic mice from the seventh generation of inbreeding were used. CAST/EiJ (CAST) males were mated to hg females to create the mapping cross (examples herein and Horvat and Medrano (1995) Genetics 139: 1737-1748). CAST mice are smaller and much leaner than C57 mice, even when the mice are on a high fat diet (York et al. (1996) Mamm. Genome 7: 677-681). Therefore, the cross is suitable to detect specific alleles interacting with hg that modify body size and composition.

A total of 75 F₁ and 1,132 F₂ mice were produced. The F₂ cross was genotyped for hg using the linked marker D10Mit41 and a marker that maps to the hg deletion, D10Mit69 (examples herein and Horvat and Medrano (1996) Genomics 36: 546-549). Mice homozygous for hg alleles at D10Mit41 and without a PCR amplification product for D10Mit69 (indicating homozygosity for the hg deletion) were considered to be of hg/hg genotype and mice homozygous for CAST alleles at D10Mit41 and amplifying for D10Mit69 were regarded as being of +/+ genotype. Using such a screen, we determined that the cross was composed of 274 +/+ mice, 596 +/hg mice and 262 hg/hg mice, which is in agreement with Mendelian segregation ratios in the F₂ population.

A second experimental cross was created by mating C57 and CAST mice. Sixty F₁, mice and 330 F₂ mice were produced. This F₂ cross was used to confirm the significance of linkage of markers identified on chromosome 2 of the F₂ cross segregating hg, by means of selective genotyping.

### Husbandry and Phenotype Determinations

Mice were weaned at 3 weeks of age. Feed (Purina 5008; 23.5% protein, 6.5% fat, 3.3 Kcal/g) and water were offered ad libitum. Mice were weighed to the nearest 0.1 g at 2, 3, 6 and 9 weeks of age and sacrificed after 9 weeks of age by cervical dislocation. Liver, spleen, and skin were removed and the carcass was frozen. To perform the chemical analysis, carcasses were thawed at room temperature. Carcass water content was determined by freeze-drying the carcasses to a constant weight. Lipid content was estimated by the carcass weight change after extraction with ether for seven days followed by acetone for five days in a Soxhlet apparatus. Body ash content was determined by incinerating the carcass in a muffle furnace at 575° C. for 16 hours. One femur bone was removed from the partially ashed carcass and measured to 0.1 mm. Only live weights at the same ages mentioned above were recorded in the C57×CAST cross.

We measured protein mass because the dry matter is considered a better estimator of dynamics of cell populations of an organ or body than their fresh weight (Graham et al. (1998) Genet. Res. 72: 247-253). We also included ash mass and femur length to have an estimation of differences in skeletal mass and size, respectively. Although we did not measure body length of the F₂ mice, Famula et al. (1988) Growth Dev. Aging 52: 145-150, demonstrated that femur length is a good predictor of body length (regression R²=0.83; uniform regression slopes between hg/hg and control mice).

### Genotyping and Linkage Analysis

In order to find QTL we followed a hierarchical search approach (Brown et al. (1994) Am. J Hum. Genet. 54: 544-552). In the first step, the hg/hg mice from the F₂ cross were genotyped with a set of 59 SSLP markers (Research Genetics, Huntsville, Ala.) covering the 19 autosomal chromosomes and chromosome X (FIG. 10). Some of these markers were chosen based on their proximity to known genes and previously identified growth QTL. The typing of markers was performed following conventional PCR and agarose gel electrophoresis methods. The linkage of these markers to loci affecting weight gain and body composition was evaluated through ANOVA, using the GLM procedure of SAS (SAS 1998). The model included marker genotype information and the fixed effects of sex, parity, litter size, and two-way interactions. The analyzed traits were body weight at 2, 3, 6 and 9 weeks of age, weight gain from 2 to 6, 6 to 9 and 2 to 9 weeks of age (G26, G69 and G29, respectively), carcass protein, carcass ash, femur length, and carcass fat percentage. The analysis of body weight at 2 and 3 weeks of age and G69 produced no significant linkage results. The results for body weight at 6 and 9 weeks of age and for G26 were almost identical to those for G29. In addition, evaluation of phenotypic data from the F₂ cross suggested that hg had a more noticeable effect on growth rates than on live weight differences (Table 1). Therefore, we report here the results corresponding to G29 and carcass composition traits.

TABLE 1
Means and standard deviations (SD) of traits measured in the
C57-hg/hg x CAST F₂ cross.

| | Females | | | | | | |
|---|---|---|---|---|---|---|---|
| | +/+ mice | | | hg/hg mice | | | |
| | n | Mean | SD | n | Mean | SD | Prob |
| Wt2 g | 138 | 8.8 | 1.3 | 130 | 8.6 | 1.5 | N.S. |
| Wt3 g | 139 | 11.5 | 1.7 | 130 | 11.5 | 1.9 | N.S. |
| Wt6 g | 134 | 17.5 | 2.0 | 129 | 20.2 | 3.1 | <0.0001 |
| Wt9 g | 130 | 19.2 | 2.3 | 123 | 23.4 | 3.3 | <0.0001 |
| G29 g | 131 | 10.4 | 2.2 | 123 | 14.8 | 3.1 | <0.0001 |
| Carcass weight g | 129 | 12.42 | 1.86 | 122 | 15.47 | 2.78 | <0.0000 |
| Carcass protein g | 128 | 2.41 | 0.34 | 121 | 2.91 | 0.51 | <0.0001 |
| Carcass ash g | 128 | 0.65 | 0.10 | 122 | 0.81 | 0.16 | <0.0001 |
| Carcass fat % | 128 | 10.4 | 4.0 | 122 | 12.8 | 5.2 | <0.0001 |
| Femur length mm | 127 | 14.5 | 0.6 | 122 | 15.4 | 0.9 | <0.0001 |
| | Males | | | | | | |
| | +/+ Mice | | | hg/hg Mice | | | |
| | n | Mean | SD | n | Mean | SD | Prob |
| Wt2 g | 135 | 9.3 | 1.4 | 130 | 8.6 | 1.4 | <0.0001 |
| Wt3 g | 135 | 12.7 | 1.9 | 130 | 12.1 | 2.2 | <0.05 |
| Wt6 g | 131 | 21.2 | 2.5 | 127 | 24.4 | 4.4 | <0.0001 |
| Wt9 g | 124 | 23.8 | 3.2 | 123 | 28.7 | 4.7 | <0.0001 |
| G29 g | 124 | 14.6 | 2.9 | 123 | 20.1 | 4.2 | <0.0001 |
| Carcass weight g | 130 | 14.99 | 2.13 | 123 | 18.36 | 3.33 | <0.0001 |
| Carcass protein g | 130 | 2.97 | 0.4 | 123 | 3.53 | 0.64 | <0.0001 |
| Carcass ash g | 130 | 0.71 | 0.09 | 123 | 0.85 | 0.15 | <0.0001 |
| Carcass fat % | 130 | 9.6 | 4.5 | 123 | 12.4 | 5.6 | <0.0001 |
| Femur length mm | 130 | 14.9 | 0.6 | 119 | 15.6 | 0.8 | <0.0001 |

Wt2-Wt9: Live weights at 2, 3, 6, and 9 weks of age, respectively.
G29: Weight gai from 2 to 9 weeks of age.

The threshold to declare significant linkage in the ANOVA was established by choosing a genome-wide P value of 0.10 and applying the Bonferroni correction for multiple comparisons (SAS 1998). Therefore, a nominal value of P 0.006 was considered indicative of linkage in the single-marker analyses. More markers were added in those chromosomes showing significant linkage in the ANOVA, for a total of 79 markers (FIG. 10). The GGT (Graphical Genotypes) software (van Berloo (1999) J. Hered. 90: 328-329) was used to create a graphical display of the genotyped chromosomes in each individual in order to assist in error checking and genotyping quality control.

In order to determine the location of a locus on a given chromosome, interval mapping was performed using regression analysis (Haleyand Knott (1992) Heredity 69: 315-324). The appropriate programs were written and run with the SAS software (SAS 1998). Before performing the final linkage analysis, the regression programs were tested with the same data set used when hg was mapped (Horvat and Medrano (1995) Genetics 139:1737-1748). The regression analysis produced almost identical results to those using Mapmaker (Lander et al. (1987) Genomics 1: 174-181) in the earlier report.

Before proceeding to the analysis, we also evaluated positions and orders of our markers using Mapmaker. The estimated distances between markers that we obtained with Mapmaker (Lander et al. (1987) Genomics 1: 174-18 1) were in agreement with the mouse consensus map (MGD 2000); therefore, we used the information from the consensus map for the analysis.

The regression models included additive (a) and dominance (d) terms, together with the effects of sex and age. Conditioning markers were included in the models to account for background genetic effects (Zeng (1994) Genetics 136: 1457-1468). These markers were selected for each trait by backward regression analysis with a probability of P<0.05. In a first step, single chromosomes were analyzed. In a second step, all significant markers were evaluated together and only those remaining after the backward selection were included in the models. Conditioning markers were omitted from the model when their corresponding chromosomes were analyzed.

The regression analysis between two markers was performed at 2 cM intervals. The results were expressed as LOD scores, LOD=4.605×Likelihood ratio test (LR), where LR=n×logₑ (RSSreduced/RSSfull) (n is the sample size, and RSSfull and RSSreduced are the Residual Sum of Squares of the complete regression model (full) and the model with the additive (a) and dominance (d) terms omitted (reduced), respectively (Haley and Knott (1992) Heredity 69: 315-324).

Empirical significance thresholds were calculated using a permutation method (Churchill and Doerge (1994) Genetics 138: 963-971). Phenotypes were permuted against genotypes and conditioning markers and the regression analyses were repeated 1,000 times. The experiment-wise significance threshold, P<0.05 or P<0.01, for each trait was established by choosing the 50th or the 10th highest LOD score across all chromosomes, respectively. The estimated experiment-wise thresholds for the interval mapping had similar values among traits and chromosomes, with extremes of 1.80 for femur length and 2.47 for carcass protein (P<0.05), and 2.65 for carcass protein and 3.25 for carcass ash (P<0.01).

The most significant markers in the hg/hg subpopulation were also typed in the +/+ subpopulation in order to verify whether the same loci were detected as QTL in the genetic background carrying the wild type allele at the hg locus. Chromosome 2 seemed to harbor genes with very strong effect on growth, both in the +/+ and hg/hg subpopulations. Therefore, we genotyped the +/+ mice with the same set of markers that we used on hg/hg mice. Interval mapping was performed as described above.

To confirm the linkage of markers D2Mit389 and D2Mit260 to QTL in chromosome 2 in an independent population, we performed selective genotyping in the F₂ cross (N=330) that did not segregate hg. Data on weight gain from 3 to 6 weeks of age was corrected for the effects of dam, litter, and parity with the GLM procedure of SAS (SAS 1998). The mice were ranked based on the adjusted data and 24 mice (12 from each sex) from the extreme ends of the F2 distribution were typed. The means of the high and low weight gain groups were 1.83 standard deviations above and 1.68 standard deviations below the population mean, respectively. For each marker a chi-square test was performed with the FREQ procedure of SAS (SAS 1998) to compare allele frequencies between the two groups.

### Results

As a relative comparison of the size of +/+ and hg/hg mice the phenotypic means of traits recorded in our F₂ population are presented in Table 1. At 2 and 3 weeks of age, there were no significant differences in weight between +/+ and hg/hg females. However, hg/hg males were smaller than +/+ mice at the same ages. At 6 and 9 weeks of age, hg/hg mice of both sexes were significantly heavier than the wild type mice. These results show that hg/hg mice grew faster than +/+ mice, especially after weaning. In fact, a significant genotype x sex interaction was detected for weight gain between 2 and 9 weeks of age. Among females, hg/hg mice gained 42% more weight than +/+ mice, whereas the difference in weight gain for males was 38%. The increased growth rate of hg/hg mice is typical of the effect of this locus (Bradford and Famula (1984) Genet. Res. 44: 293-308).

At sacrifice, hg/hg mice had heavier carcasses than +/+ mice. The carcasses of hg/hg mice had more protein and ash, a longer femur, and a higher fat content than the carcasses of +/+ mice (Table 1).

Eight chromosomes harboring significant markers where identified with ANOVA and then analyzed by interval mapping. Eight loci were identified through interval mapping, which were designated Q2Ucd1, Q2Ucd2, Q5Ucd1, Q8Ucd1, Q9Ucd1, Q9Ucd2, Q11Ucd1 and Q17Ucd1 (Table 2). Markers on chromosomes 13 and X were identified as significant in the ANOVA but did not reach the minimum significance thresholds by interval mapping. No significant sex-specific effects were detected in our scan.

Significant loci identified in hg/hg mice can be arbitrarily placed in four groups. Group 1: Loci in chromosomes 2 and 8 (Q2Ucd1 and Q8Ucd1) which affected only the growth rate of F₂ mice. Group II: Loci on chromosomes 2 and 11 (Q2Ucd2 and Q11Ucd1) that affected growth rate and carcass lean mass (protein and ash). Although we conducted independent analyses for each trait, the consistency in the location of these two loci in the different analyses made us consider that the same locus was affecting more than one trait, which is highly suggestive of pleiotropy. Group III: Loci on chromosome 9 (Q9Ucd2) that modified femur length and chromosome 17 (Q17Ucd1) that modified carcass lean mass (protein and ash) and femur length. These two loci had no effect on growth rate. For chromosome 17, we also assumed that a single locus was affecting two different traits. Group IV: Loci on chromosomes 5 and 9 (Q5Ucd1 and Q9Ucd1) that modified carcass fat content. QTL displayed various gene action modes (Table 2) from additivity (e.g., Q17Ucd1-p), dominance (e.g., Q2Ucd1-femur), partial dominance (e.g., Q11Ucd1-p) to overdominance (e.g., Q2Ucd2-wg29).

TABLE 2
Most likely locations and effects of loci detected in the hg/hg subpopulation.

| | | | Location | | Additivity | Dominance | |
|---|---|---|---|---|---|---|---|
| Trait | Locus¹ | Chrom. | (Cm) | LOD² | (SE) | ((SE) | % Vp³ |
| Weight gain | Q2Ucd1-wg29 | 2 | 31 | 3.75 | 1.174 | 0.841 | 4.2 |
| | | | | | (0.327) | (0.463) | |
| 2-9 wk (g) | Q2Ucd2-wg29 | 2 | 61 | 7.43 | 1.309 | 2.286 | 10.4 |
| | | | | | (0.319) | (0.509) | |
| | Q8Ucd1-wg29 | 8 | 45 | 3.01 | 0.892 | 1.397 | 4.3 |
| | | | | | (0.328) | (0.512) | |
| | Q11Ucd1-wg29 | 11 | 46 | 3.41 | 1.283 | 0.320 | 4.1 |
| | | | | | (0.324) | (0.491) | |
| Carcass | Q2Ucd2-p | 2 | 63 | 4.89 | 0.165 | 0.272 | 7.6 |
| | | | | | (0.043) | (0.079) | |
| Protein (g) | Q11Ucd1-p | 11 | 46 | 5.01 | 0.206 | 0.111 | 5.7 |
| | | | | | (0.042) | (0.064) | |
| | Q17Ucd1-p | 17 | 46 | 4.77 | 0.231 | −0.054 | 6.5 |
| | | | | | (0.048) | (0.069) | |
| Carcass | Q2Ucd2-ash | 2 | 63 | 4.34 | 0.037 | 0.062 | 6.9 |
| | | | | | (0.011) | (0.017) | |
| Ash (g) | Q11Ucd1-ash | 11 | 50 | 3.18 | 0.039 | 0.026 | 3.9 |
| | | | | | (0.010) | (0.015) | |
| | Q17Ucd1-ash | 17 | 48 | 3.67 | 0.048 | −0.009 | 4.9 |
| | | | | | (0.011) | (0.015) | |
| Carcass | Q5Ucd1-fp | 5 | 38 | 2.46 | −1.642 | 0.270 | 6.2 |
| | | | | | (0.492) | (0.766) | |
| Fat (%) | Q9Ucd1-fp | 9 | 10 | 5.83 | −2.347 | 0.562 | 12.5 |
| | | | | | (0.454) | (0.744) | |
| Femur | Q2Ucd2- | 2 | 59 | 2.72 | 0.202 | 0.207 | 4.2 |
| | femur | | | | (0.068) | (0.107) | |
| length (mm) | Q9Ucd2- | 9 | 20 | 6.34 | 0.365 | 0.224 | 10.7 |
| | femur | | | | (0.072) | (0.112) | |
| | Q17Ucd1- | 17 | 48 | 3.51 | 0.305 | −0.087 | 6.6 |
| | femur | | | | (0.071) | (0.096) | |

¹Locus nomenclature: Q for QTL, chromosome #, Ucd for University of California, a consecutive number for each QTL on a chromosome, and a letter code referring to the Phenotype. QTL affecting more than one trait are distinguished by the phenotype code.
²LOD scores were significant at P < 0.01, with the exception of LODs for Q5Ucdl-fp and Q2Ucd2-femur that were significant the P < 0.05.
³% Vp, % of Phenotypic Variance explained by the QTL. % Vp = VG * 100/Vp, where VG = ½a2 + ¼d2.

The loci affecting growth rate and carcass lean mass individually explained 4.1% to 10.4% of the phenotypic variance on weight gain in the hg/hg subpopulation. The locus Q2Ucd2 had very significant effects on all the studied traits with the exception of fat percentage. On the other side, the two significant loci Q5Ucd1 and Q9Ucd1 explained 6.2% and 12.5% of the phenotypic variance for fat content in the hg/hg subpopulation, respectively (Table 2). Interestingly, the C57 alleles have a positive additive effect for all the identified loci with the exception of loci Q5Ucd1 and Q9Ucd1, for which the CAST alleles were responsible for increasing carcass fat percentage.

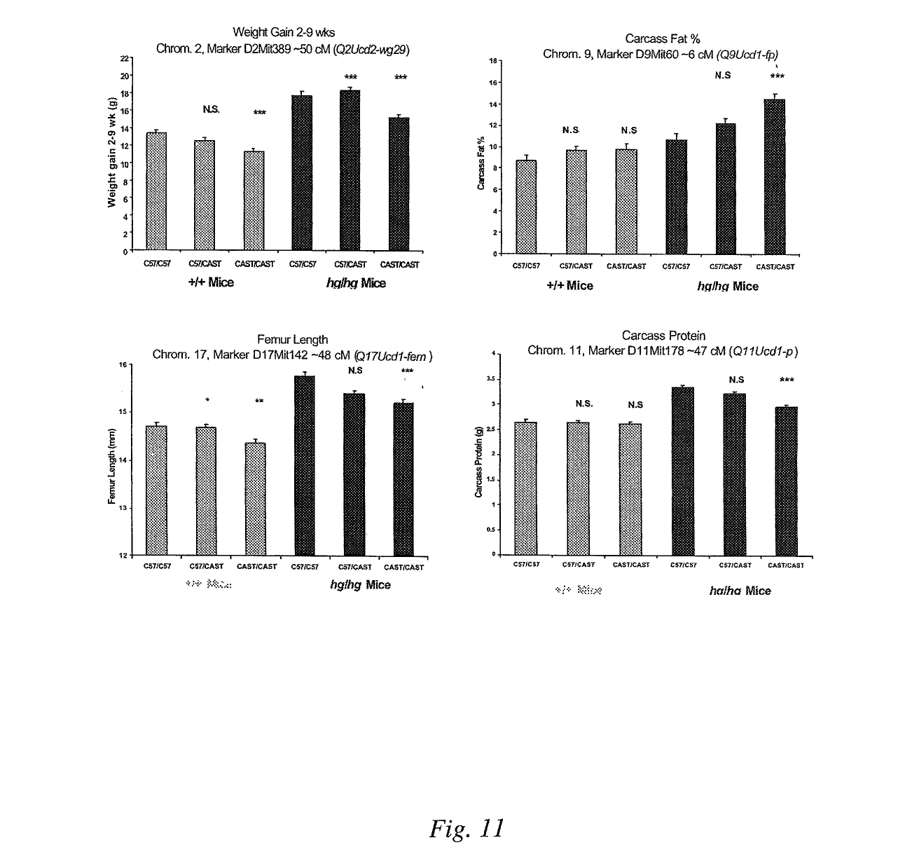

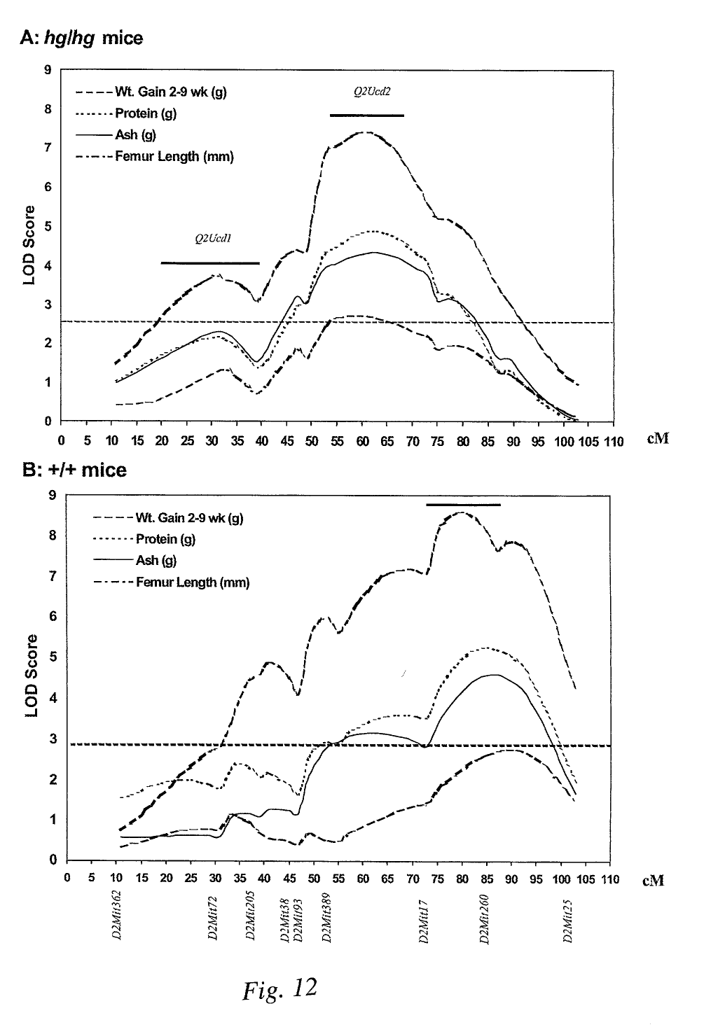

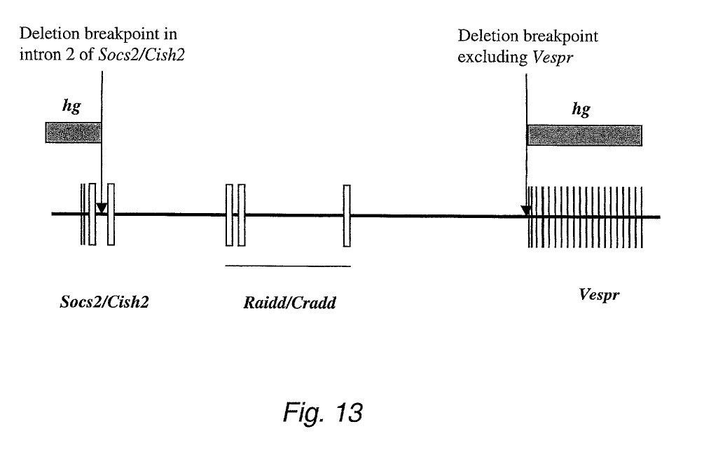

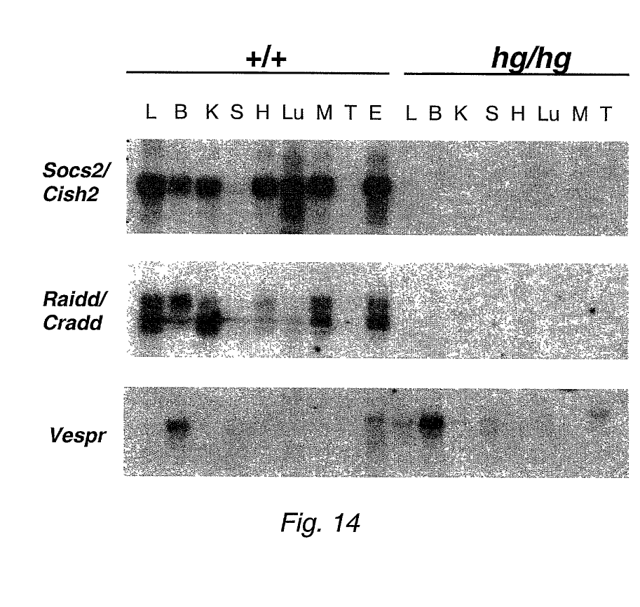

In order to identify modifiers of hg, we genotyped the marker that was closest to the significant QTL in the hg/hg group. Also in the +/+ mice of the F₂ cross. four markers showed statistically significant interactions in the ANOVA (nominal P<0.05) (FIG. 11). Interestingly, these markers are examples of three different types of genetic interaction. Marker D2Mit389 on chromosome 2 (close to Q2Ucd2) had an additive effect on weight gain on +/+ mice, but became dominant in the presence of hg. The locus on chromosome 17 (Q17Ucd1) modifying femur length, which had an additive effect in hg/hg mice, tended to be dominant in +/+ mice. Loci on chromosomes 9 and 11 (Q9Ucd1 and Q11Ucd1) that affected carcass fatness and carcass protein mass in an additive manner in the hg/hg mice had no significant effects in +/+ mice.

In view of the importance of the effect of Q2Ucd2 on growth rate and carcass lean mass of hg/hg mice, we genotyped the +/+ mice with the same markers of chromosome 2 and performed the interval mapping for growth rate (G29) and carcass composition traits (FIG. 12). It can be seen that the LOD curves have distinctive patterns in each subpopulation and that the highest LOD scores in each case corresponded to different positions on the chromosome. In +/+ mice, a very significant locus affecting growth rate and carcass lean mass was detected distal to the location of Q2Ucd2.

In order to confirm the existence of the two important loci found on chromosome 2 of hg/hg and +/+ mice, we selectively genotyped a C57×CAST F2 cross with markers D2Mit389 and D2Mit260. For D2Mit389, the frequency of the C57 allele in the groups of high and low weight gain were 0.65 and 0.33, respectively (Chi-square =10.3, P<0.0013, n=24), whereas the corresponding frequencies for D2Mit260 were 0.65 and 0.29, respectively (Chi-square=12.9, P<0.0003, n=24). These results indicate that both markers were linked to QTL and that it was not an artifact of the F₂ cross-segregating hg.

### Discussion

It has been suggested that in terms of genetic analysis, our knowledge about the genome is progressing faster than our understanding of the phenotype, a situation that has been addressed as a “phenotype gap” (Graham et al. (1998) Genet. Res. 72: 247-253). Given the complexity of the regulation of animal growth, a good characterization of the phenotype, and novel strategies for genetic analysis would help in understanding the regulation of growth and body size. In this study, we have contributed to that understanding by describing the influence of the genetic background on a major locus affecting growth.

Our genome-wide scan identified eight loci responsible for the differences in growth among hg/hg mice. The traits that we measured allowed us to distinguish loci that affected growth rate from those affecting body size and carcass composition. Growth rate and body size have a positive genetic correlation (Bishop and Hill (1985) Genet. Res. 46: 57-74); however, we found loci that affected both traits independently. Although we were primarily interested in the influence of hg on linear growth, we included the analysis of body composition based on previous knowledge about the variability between CAST and C57, not only in size and body composition (York et al. (1996) Mamm. Genome 7: 677-681). These distinguishing features between lines made the C57×CAST cross an ideal material for evaluating the effects of a major locus affecting growth.

In our search for modifiers we detected significant two-way interactions between four loci and hg (Q2Ucd2, Q9Ucd1, Q11Ucd1, Q17Ucd1). The comparison of means between the hg/hg and +/+ subpopulations for selected markers proved that not all the loci that modulated the effects of hg on growth were present in the +/+ F₂ mice. Additionally, some loci that were significant in both backgrounds had a different type of genetic action depending on the background. This result emphasizes the relevance of epistasis in the genetic regulation of growth in mammals (Routman and Cheverud (1997) Evolution 51: 1654-1662).

A large number of growth QTL have been reported in the literature. We found agreement in the position of QTL in hg/hg mice and other QTL affecting growth and body composition, which could imply that the same genes are being detected. Although we list below the comparisons of QTL positions between our cross and other crosses, we would like to make a cautionary note that such comparisons are problematic and may be questionable. It has been shown (Keightley and Knott (1999) Genet. Res. 74: 323-328) that getting a statistically significant correlation between two QTL mapping experiments (i.e. that the same QTL segregates in two crosses) would be unlikely especially for cases where the variation in the trait is explained by a large number of QTL and where experimental populations are not closely related, which is the case with the experiments compared here.

Cheverud et al. (1996) Genetics 142: 1305-1319, reported a QTL for early weight gain (1 to 3 weeks of age) on chromosome 2 in the LG/J×SM/J cross, in a similar position to Q2Ucd2. Loci affecting 6-week weight on chromosomes 2 and 11 were reported by Brockmann et al. (1998) Genetics 150, 369-381 in the DUK×DU6 cross, in the corresponding regions to where we mapped Q2Ucd2 and Q11Ucd1, respectively. In addition, a significant locus in the vicinity of Q11Ucd1 was identified by Keightley et al. (1996) Genetics 142: 227-235, in a line selected for high 6-week weight. We were not able to find information on other growth QTL mapping to the locations of Q2Ucd1, Q8Ucd1 or Q17Ucd1.

A QTL affecting adult body weight in a C57×CAST cross has been mapped to the same region in chromosome 2 where we found a very significant QTL in +/+ mice (region around 80-90 cM) (Mehrabian et al. (1998) J. Clin. Invest. 101: 2485-2496). A very large confidence interval reported for the location of that locus, together with the pattern of LOD scores along the chromosome, suggest that a second locus, probably at the same location of Q2Ucd2, could have affected body weight in that experiment. Also, an adult body weight QTL mapping to the distal region of chromosome 2 has been identified in the NZB/BINJ×SM/J cross (Lembertas et al. (1997) J. Clin. Invest. 100: 1240-1247).

Results of genomic scans for obesity QTL on C57×CAST crosses have been reported (York et al. (1996) Mamm. Genome 7: 677-681; Lembertas et al. (1997) J. Clin. Invest. 100: 1240-1247). Our loci Q5Ucd1 and Q9Ucd1 were not detected in those experiments. Because high fat diets were used in the two cited experiments, their results are not strictly comparable to ours. However, we did not detect those two loci as significant in the +/+ subpopulation either. There is evidence in the literature of loci affecting carcass composition in similar locations to Q5Ucd1 and Q9Ucd1. Taylor and Phillips (1999) Mamm. Genome 10: 963-968suggested the existence of a putative obesity QTL in the central portion of chromosome 5 in a 129/Sv×EL/Suz cross. Also, the locus Obq5 identified in a C57×KK/HlLt cross, which affected several adiposity-related traits mapped close to Q9Ucd1. Together, these results indicate that hg produced metabolic changes beyond the effects on growth rate and body size. Interestingly, hg/hg F2 mice were on average fatter than their wild type counterparts (Table 1). Previous experiments comparing the body composition of hg/hg and control mice found differences in carcass composition of the magnitude reported here (Calvert et al. (1984) J. Anim. Sci. 59: 361-365). The CAST alleles at Q5Ucd1 and Q9Ucd1 increased carcass fat content, but only in hg/hg mice (Table 2). Therefore, this particular effect on body composition could involve specific alleles from CAST origin interacting with hg. This result is not surprising. It has been confirmed for both body weight (Cheverud et al. (1996) Genetics 142: 1305-1319) and body composition (Mehrabian et al. (1998) J. Clin. Invest. 101: 2485-2496) that alleles that increase the phenotypic mean of a trait in a mapping cross may come from the parental inbred line with the lower phenotypic mean for that trait.

Among the QTL detected in hg/hg mice there are loci that influenced both growth rate and final body size, and loci that affected each trait independently (Table 2). The final body size of an animal is mostly a function of its cell mass, which in turn results from the product of cell number and cell size (Conlon and Raff (1999) Cell 96: 235-244). Although there is agreement about the existence of a genetic regulation of those parameters, little is known about how the changes in cell number and size that lead to a change in body mass are coordinated with time (Su and O'Farrell (1998) Curr. Biol. 8: 8687-689; Conlon and Raff (1999) Cell 96: 235-244). Our results confirm that although there are genetic factors underlying a connection between final body size and the time taken to achieve it (Webster AJF (1989) Anim. Prod. 48, 249-269), there are genes that act independently on each of those variables, probably through alterations of the dynamics of cell proliferation, cell enlargement, or both (Su and O'Farrell (1998) Curr. Biol. 8: 8687-689; Conlon and Raff (1999) Cell 96: 235-244).

The regulation of growth rate and ultimately body size can be regarded to be a result of the balance between growth promoting and growth inhibiting factors (Efstratiadis (1998) Int. J. Dev. Biol. 42: 955-976). The recent discovery of myostatin, a negative regulator of muscle mass, is consequent with the existence of growth inhibitors (McPherron et al. (1997) Nature 387: 83-90; Lee and McPherron (1999) Curr. Opin. Genet. Dev. 9: 604-607). The product of the hg locus could be one of those growth-inhibiting factors and its modifiers could well be genes associated with inhibitory pathways that set limits to the growth process. Recently the hg phenotype has been identified as resulting from a lack of expression of the suppressor of cytokine signaling 2 (Socs2 or Cish2; see Examples herein). Also, identification of modifiers of hg, detected here, will be of value in identifying the genes involved in the functional pathways leading to variation in phenotypic size in different genetic backgrounds. For example, high levels of IGF-I have been consistently reported in hg/hg mice (Medrano et al. (1991) Genet. Res. 58: 67-74; Reiser et al. (1996) Am. J. Physiol. 271: 8696-703). At least some of the modifiers that we report here could ultimately be genes involved in pathways influenced by IGF-I. It is also possible that interactions between the hg allele (lack of Socs2) and modifiers of hg are responsible for some of the reproductive problems that have been documented in this mutation (Cargill et al. (1999) Biol. Reprod. 61: 283-287). It would be useful, therefore, to fine map modifiers of hg and ultimately positionally clone them.

### Example 4

### High Growth Mouse Research Update

The high growth mutation was found in a strain of mice selected for high 3 to 6 week post-weaning weight gain. The mutation causes a major increase in growth (30-50% in homozygotes) that is proportional in all tissues and organs and does not result in obesity. High growth (HG) mice are on average 13% leaner but much higher in weight than controls at the same age. This effect is particularly noticeable when control and HG mice are fed high-energy diets. The mutation alters energy metabolism by increasing efficiency of growth and/or decreasing maintenance energy requirements. The effects of the mutation are detected early in development, manifested by delayed muscular cell fusion and an increase in muscle fiber number (Summers, and Medrano (1994) Growth, Devel & Aging 58: 135-148; Summers and Medrano (1996) J. Experimental Biology and Medicine 214: 380-385). Interestingly, High Growth mice have lower concentrations of Growth Hormone (GH) but much higher concentrations of Insulin-like Growth Factor I (IGF-I) in their plasma than normal mice (Medrano et al. (1991) Genetical Research 58: 67-74; Reiser et al. (1996) American journal of physiology-regulatory integrative and comparative Physiology 40: R696-R703; Corva, and Medrano (2000) Physiological Genomics 3:17-23).

We initially localized hg by interval mapping to the distal half of mouse chromosome 10 and confirmed its location by test-crossing (see Examples herein and Horvat and Medrano (1995) Genetics 139: 1737-1748). This was followed by the development of new fine mapping markers (see Examples herein and Horvat and Medrano (1996) Genomics 36: 546-549) and by the mapping of hg to a 500-kb deletion. A YAC/BAC contig map spanning the deletion was developed and the Raidd/Cradd gene was identified within the deletion as a potential candidate for hg (see Examp sherein and Horvat and Medrano (1998) Genomics 54:159-164). We have shotgun sequenced 6 BAC clones spanning the hg deletion to identify potential transcripts. Our sequence covers approximately 650,000 bp and it is composed of 13 contigs (see e.g., SEQ ID NO: 20). Clones spanning the ends of these contigs are currently being sequenced to close the gaps.

A brief description of the genes in the high growth region is included. FIG. 14 shows the pattern of expression of these genes in control and HG mice.

### Socs2/Cish2 (suppressor of cytokine signaling 2/cytokine-inducible SH2-containing protein 2)

Socs2 is the name of the gene according to the human nomenclature and Cish2 is the name according to the mouse nomenclature: Socs2/Cish2 is a member of a gene family of negative regulators of cytokines. Citokines are secreted proteins that interact with specific cell surface receptors, triggering cytoplasmic signal transduction pathways that transmit the signal to the nucleus and initiate changes in transcription.

Socs2 is not expressed in hg mice, eliminating a negative regulation of signaling from growth-promoting cytokines. Our earlier observations that HG mice have low plasma levels of growth hormone with no pulsatile effect, as well as high levels of IGF-1, are consistent with the effect that the lack of Socs2 may have as a regulator of growth hormone and IGF-1 signaling. Socs2 has been shown to act as a suppressor of growth hormone signaling and to directly interact with the IGF-1 receptor.

For a growth control gene with an inhibitory function like Socs2, it is expected that the lack of expression has a growth-promoting effect, and overexpression has growth-inhibiting effect. Therefore, manipulation of Socs2 protein expression is expected to be useful in domestic animals as a strategy for improving animal growth, and in human medicine for treating growth disorders.

### Raidd/Cradd (RIP-Associated ICDH/CED-3-Homologous Protein with DD/Caspase and RIP Adapter with DD)

Apotosis, or programmed cell death, occurs during normal cellular differentiation and development of multicellular organisms. Apotosis is induced by certain cytokines including TNF, TNFR-1 and Fas. The death signals are transduced by a group of DD (death domain)-containing adapter molecules. RAIDD is one of these adapter molecules, which interacts with RIP and caspase-2 to transduce death signals.

Raidd/Cradd originally appeared as a very good candidate for the high growth phenotype. As stated above, the main characteristic of HG is the increase in postnatal growth, resulting in an increase in muscle mass and in the size of tissues and organs. Therefore, HG mice are most likely bigger because they have more cells, not because their cells are larger. These differences in cell number may have been due to a perturbed apoptosis program caused by a lack of function of Raidd/Cradd in the apoptotic-signaling pathway. However, Raidd/Cradd knockout mice do not exhibit increased growth or any obvious phenotypic difference. Therefore, questions remain on the function and the role this gene may have in relation to the high growth phenotype and if it interacts with Socs2/Cish2 in the resulting phenotype.

### Vespr (Viral Encoded Semaphorin Receptor)

Semaphorins and their receptors are molecules that act as mediators of immune function. Semaphorins comprise a large family of secreted and transmembrane proteins, some of which deliver guidance cues to migrating axons during development. Semaphorins have also been identified on the surface of hematopoietic cells and in the genome of certain lytic viruses. In the immune system, VESPR, a member of the Plexin family, is a receptor for a viral-encoded Semaphorin. (Weinberg et al. (1998) Cell 95: 903-916). The gene is primarily expressed in brain tissue, in both HG and control mice. Mouse Vespr is a large gene and protein (mRNA 4725 bp, protein 1574 amino acids). The mRNA sequence of the gene has been published in GenBank (accession # AF190578). FIG. 14: mRNA Northern blot showing the lack of expression of Socs2/Cish2 and Raidd/Cradd in various tissues (L, liver; B, brain, K, kidney; H, heart; Lu, lung; M, muscle; T, testis; E, 13d. embryo) in high growth (hg/hg) mice and the positive expression of Vespr in comparison to control mice (+/+).

We have also completed a genome scan in F₂ hg/hg mice to identify contributing QTL to the high growth effect (see Examples herein). Eight significant loci were identified through interval mapping. Loci on chromosomes 2 and 8 affected the growth rate, loci on chromosomes 2 and 11 affected growth rate and carcass lean mass (protein and ash). A locus on chromosome 9 modified femur length and another one in chromosome 17 affected both carcass lean mass and femur length. Loci on chromosomes 5 and 9 modified carcass fat content. Significant interactions between hg and other growth QTL were identified, which were detected as changes in gene action (additive or dominant) and in allele substitution effects.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An isolated nucleic acid molecule encoding a gene product that, when knocked out, results in a high growth (hg) phenotype.

2. The nucleic acid of clam 1, wherein said nucleic acid comprises the nucleotide sequence of SEQ ID NO: 9.

3. The nucleic acid of claim 1, wherein said nucleic acid is present in a vector.

4. The nucleic acid of claim 1, wherein said nucleic acid is a DNA.

5. The nucleic acid of claim 1, comprising the nucleic acid or the complement of the nucleic acid of SEQ ID NO: 9.

6. A cell transfected with a nucleic acid molecule encoding a gene product that, when knocked out, results in a high growth (hg) phenotype.

7. The cell of claim 4, wherein said cell is a mammalian cell.

8. A method of producing an animal characterized by a high growth phenotype, said method comprising inhibiting expression of a Socs2 gene.

9. The method of claim 8, wherein said inhibiting is by disrupting said gene by homologous recombination with a nucleic acid that undergoes homologous recombination with a Socs2 gene and introduces a disruption in said Socs2 gene.

10. The method of claim 9, wherein said nucleic acid encodes a selectable marker.

11. The method of claim 10, wherein said selectable marker is as neo or a hyg gene or cDNA.

12. A knockout mammal, said mammal comprising cells containing a recombinantly introduced disruption in a Socs2 gene, wherein said disruption results in said knockout mammal exhibiting decreased levels of SOCS2 protein as compared to a wild-type mammal.

13. The knockout mammal of claim 12, wherein said mammal displays a high growth (hg) phenotype.

14. The knockout mammal of claim 12, wherein said mammal is selected from the group consisting of an equine, a bovine, a rodent, a porcine, a lagomorph, a feline, a canine, a murine, a caprine, an ovine, and a non-human primate.

15. The knockout mammal of claim 12, wherein, wherein the disruption is selected from the group consisting of an insertion, a deletion, a frameshift mutation, a substitution, and a stop codon.

16. The knockout mammal of claim 15, wherein, wherein said disruption comprises an insertion of an expression cassette into the endogenous Socs2 gene.

17. The knockout mammal of claim 16, wherein, wherein said disruption comprises an expression cassette comprising a selectable marker.

18. The knockout mammal of claim 16, wherein the expression cassette comprises a neomycin phosphotransferase gene operably linked to at least one regulatory element.

19. The knockout mammal of claim 12, wherein said disruption is in a somatic cell.

20. The knockout mammal of claim 12, wherein said disruption is in a germ cell.

21. The knockout mammal of claim 12, wherein the mammal is homozygous for the disrupted Socs2 gene.

22. The knockout mammal of claim 12, wherein the mammal is heterozygous for the disrupted Socs2 gene.

23. The knockout mammal of claim 12, wherein said mammal further comprises a second recombinantly disrupted gene.

24. The knockout mammal of claim 23, wherein said second gene comprises a disruption that prevents the expression of a functional polypeptide from said disrupted second gene.

25. The knockout mammal of claim 23, wherein the mammal is homozygous for said disrupted second gene.

26. The knockout mammal of claim 23, wherein the mammal is heterozygous for said disrupted second gene.

27. A knockout rodent comprising a recombinantly introduced disruption in an endogenous SOCS2 gene (Socs2) wherein said disruption results in said knockout rodent exhibiting decreased levels of SOCS2 protein as compared to a wild-type rodent.

28. The knockout rodent of claim 27, wherein said mammal displays a high growth (hg) phenotype.

29. The knockout rodent of claim 27, wherein, wherein the disruption is selected from the group consisting of an insertion, a deletion, a frameshift mutation, a substitution, and a stop codon.

30. The knockout rodent of claim 27, wherein, wherein said disruption comprises an insertion of an expression cassette into the endogenous Socs2 gene.

31. The knockout mammal of claim 30, wherein, wherein said disruption comprises an expression cassette comprising a selectable marker.

32. The knockout mammal of claim 30, wherein the expression cassette comprises a neomycin phosphotransferase gene operably linked to at least one regulatory element.

33. The knockout rodent of claim 27, wherein said disruption is in a somatic cell.

34. The knockout rodent of claim 27, wherein said disruption is in a germ cell.

35. The knockout rodent of claim 27, wherein the rodent is homozygous for the disrupted Socs2 gene.

36. The knockout rodent of claim 27, wherein the rodent is heterozygous for the disrupted Socs2 gene.

37. A method of screening for an agent that modulates expression of a high growth (hg) phenotype, said method comprising:
contacting a cell comprising a Socs2 gene with a test agent; and
detecting a change in the expression or activity of a Socs2 gene product as compared to the expression or activity of a Socs2 gene product in a cell that is contacted with the test agent at a lower concentration, where a difference in the expression or activity of Socs2 in the contacted cell and the cell that is contacted with the lower concentration indicates that said agent modulates expression of a high growth phenotype.

38. The method of claim 37, wherein said lower concentration is the absence of said test agent.

39. The method of claim 37, wherein the amount of Socs2 gene product is detected by detecting Socs2 mRNA in said sample.

40. The method of claim 39, wherein said level of Socs2 mRNA is measured by hybridizing said mRNA to a probe that specifically hybridizes to a Socs2 nucleic acid.

41. The method of claim 40, wherein said hybridizing is according to a method selected from the group consisting of a Northern blot, a Southern blot using DNA derived from the Socs2 RNA, an array hybridization, an affinity chromatography, and an in situ hybridization.

42. The method of claim 40, wherein said probe is a member of a plurality of probes that forms an array of probes.

43. The method of claim 39, wherein the level of Socs2 mRNA is measured using a nucleic acid amplification reaction.

44. The method of claim 37, wherein the amount of Socs2 gene product is detected by detecting the level of a Socs2 protein in said biological sample.

45. The method of claim 37, wherein said detecting is via a method selected from the group consisting of capillary electrophoresis, a Western blot, mass spectroscopy, ELISA, immunochromatography, and immunohistochemistry.

46. The method of claim 37, wherein said cell is cultured ex vivo.

47. The method of claim 37, wherein said test agent is contacted to an animal comprising a cell containing the Socs2 nucleic acid or the Socs2 protein.

48. A method of prescreening for an agent that alters the expression of a high growth phenotype, said method comprising:
i) contacting a Socs2 nucleic acid or a Socs2 protein with a test agent; and
ii) detecting specific binding of said test agent to said Socs2 protein or nucleic acid.

49. The method of claim 48, further comprising recording test agents that specifically bind to said Socs2 nucleic acid or protein in a database of candidate agents that alter hg phenotype development.

50. The method of claim 48, wherein said test agent is not an antibody.

51. The method of claim 48, wherein said test agent is not a protein.

52. The method of claim 48, wherein said test agent is not a nucleic acid.

53. The method of claim 48, wherein said test agent is a small organic molecule.

54. The method of claim 48, wherein said detecting comprises detecting specific binding of said test agent to said Socs2 nucleic acid.

55. The method of claim 54, wherein said binding is detected using a method selected from the group consisting of a Northern blot, a Southern blot using DNA derived from a Socs2 RNA, an array hybridization, an affinity chromatography, and an in situ hybridization.

56. The method of claim 48, wherein said detecting comprises detecting specific binding of said test agent to said Socs2 protein.

57. The method of claim 48, wherein said detecting is via a method selected from the group consisting of capillary electrophoresis, a Western blot, mass spectroscopy, ELISA, immunochromatography, and immunohistochemistry.

58. The method of claim 48, wherein said test agent is contacted directly to the Socs2 nucleic acid or to the Socs2 protein.

59. The method of claim 48, wherein said test agent is contacted to a cell containing the Socs2 nucleic acid or the Socs2 protein.

60. The method of claim 59, wherein said cell is cultured ex vivo.

61. The method of claim 48, wherein said test agent is contacted to an animal comprising a cell containing the Socs2 nucleic acid or the Socs2 protein.

62. An isolated nucleic acid comprising a nucleic acid selected from the group consisting of:
a nucleic acid that specifically hybridizes to a nucleic acid selected from the group consisting of SEQ ID NO:2, and SEQ ID NO: 9 under stringent conditions;
nucleic acid comprising a nucleic acid sequence selected from the group consisting of SEQ ID NO:2, and SEQ ID NO: 9.

63. The nucleic acid of claim 62, wherein said nucleic acid is at least 15 nucleotides in length.

64. A polypeptide comprising a polypeptide encoded by a nucleic acid of claim 62.

65. An antibody that specifically binds a polypeptide of claim 64.

66. A nucleic acid for disrupting a SOCS2 gene (Socs2), said nucleic acid comprising:
SOCS2 gene sequences that undergo homologous recombination with an endogenous SOCS2 gene: and
a nucleic acid sequence that, when introduced into a SOCS2 gene inhibits the expression of said SOCS2 gene.

67. The nucleic acid of claim 66, wherein said nucleic acid when introduced into a SOCS2 gene creates a disruption selected from the group consisting of an insertion, a deletion, a frameshift mutation, and a stop codon.

68. The nucleic acid of claim 66, wherein the disruption comprises the insertion of an expression cassette into the endogenous SOCS2 gene.

69. The nucleic acid of claim 66, wherein the expression cassette comprises a selectable marker.

70. The nucleic acid of claim 66, wherein said nucleic acid comprises Socs2 nucleic acid sequences flanking a nucleic acid encoding a Socs2 disruption.

71. The nucleic acid of claim 66, wherein said nucleic acid is present in a vector.

72. An animal cell comprising a recombinantly introduced disruption in an endogenous SOCS2 gene (Socs2) wherein said disruption results in said cell exhibiting decreased levels of SOCS2 protein as compared to a wild-type cell.

73. The cell of claim 72, wherein said cell of a animal is selected from the group consisting of a chicken, a turkey, a duck, a goose, an equine, a bovine, a rodent, a porcine, a lagomorph, a feline, a canine, a murine, a caprine, an ovine, and a non-human primate.

74. The cell of claim 72, wherein the cell is a rodent cell.

75. The cell of claim 72, wherein the disruption is selected from the group consisting of an insertion, a deletion, a frameshift mutation, and a stop codon.

76. The cell of claim 72, wherein the disruption comprises an insertion of an expression cassette into the endogenous SOCS2 gene.
